(19) United States
(12) Patent Application Publication
(10) Pub. No.: US 2005/0002968 A1
(43) Pub. Date: Jan. 6, 2005

(54) Flavivirus vaccines

(76) Inventors: Thomas P. Monath, Harvard, MA (US); Farshad Guirakhoo, Melrose, MA (US); Juan Arroyo, Rockville, MD (US); Konstantin Pugachev, Natick, MA (US)

Correspondence Address: CLARK & ELBING LLP, 101 FEDERAL STREET, BOSTON, MA, 02110, US

(21) Appl. No.: 10/789,842

(22) Filed: Feb. 27, 2004

Related U.S. Application Data
(63) Continuation-in-part of application No. 10/345,036, filed on Jan. 15, 2003.
(60) Provisional application No. 60/348,949, filed on Jan. 15, 2002.
(60) Provisional application No. 60/385,281, filed on May 31, 2002.

(51) Int. Cl. A61K 39/12; A61K 39/193; C12N 7/00

(52) U.S. Cl. 424/218.1; 435/235.1

(57) ABSTRACT

The invention provides attenuated flavivirus vaccines and methods of making and using these vaccines.

This application is a continuation-in-part of U.S. Ser. No. 10/345,036, filed Jan. 15, 2003, which claims priority from U.S. Provisional Patent Application Serial Nos. 60/348,949, filed Jan. 15, 2002, and 60/385,281, filed May 31, 2002, the contents of each of which are incorporated herein by reference.

### FIELD OF THE INVENTION

This invention relates to flavivirus vaccines.

### BACKGROUND OF THE INVENTION

Flaviviruses are small, enveloped, positive-strand RNA viruses that are mostly transmitted by infected mosquitoes or ticks. Several flaviviruses, such as yellow fever, dengue, Japanese encephalitis, tick borne encephalitis, and West Nile viruses, pose current or potential threats to global public health. Yellow fever virus, for example, has been the cause of epidemics in certain jungle locations of sub-Saharan Africa, as well as in some parts of South America. Although many yellow fever infections are mild, the disease can also cause severe, life-threatening illness. The initial or acute phase of the disease state is normally characterized by high fever, chills, headache, backache, muscle aches, loss of appetite, nausea, and vomiting. After three to four days, these symptoms disappear. In some patients, symptoms then reappear, as the disease enters its so-called toxic phase. During this phase, high fever reappears and can lead to shock, bleeding (e.g., bleeding from the mouth, nose, eyes, and/or stomach), kidney failure, and liver failure. Indeed, liver failure causes jaundice, which is yellowing of the skin and the whites of the eyes, and thus gives “yellow fever” its name. About half of the patients who enter the toxic phase die within 10 to 14 days. However, persons that recover from yellow fever have lifelong immunity against reinfection. The number of people infected with yellow fever virus over the last two decades has been increasing, with there now being about 200,000 yellow fever cases, with about 30,000 deaths, each year. The re-emergence of yellow fever virus thus presents a serious public health concern.

Dengue (DEN) virus is the cause of a growing public health problem worldwide due to a dramatic growth in its prevalence. The disease is now endemic in more than 100 countries in the Americas, Southern Europe, Asia, and Australia. Two and a half billion people, two-fifths of the world's population, are now at risk of infection. Over 50 million infections and 24,000 deaths due to dengue are recorded annually. Dengue virus has four distinct but closely related serotypes, serotypes 1-4. Infection with one serotype generally induces life long immunity against that serotype, but only confers a transient protection against the other three. Worse than providing only transient protection, sequential infection with different serotypes has been found to increase the risk of dengue hemorrhagic fever (DHF) and dengue shock syndrome (DSS), which are potentially lethal complications of the disease. Thus, protection against all four serotypes is necessary, as protection against one or two serotypes may actually enhance the risk of subsequent infections with other serotypes, therefore putting subjects at risk of DHF/DSS.

Flaviviruses, including yellow fever virus and dengue virus, have two principal biological properties responsible for their induction of disease states in humans and animals. The first of these two properties is neurotropism, which is the propensity of the virus to invade and infect nervous tissue of the host. Neurotropic flavivirus infection can result in inflammation and injury of the brain and spinal cord (i.e., encephalitis), impaired consciousness, paralysis, and convulsions. The second biological property of flaviviruses is viscerotropism, which is the propensity of the virus to invade and infect vital visceral organs, including the liver, kidney, and heart. Viscerotropic flavivirus infection can result in inflammation and injury of the liver (hepatitis), kidney (nephritis), and cardiac muscle (myocarditis), leading to failure or dysfunction of these organs. Neurotropism and viscerotropism appear to be distinct and separate properties of flaviviruses.

Some flaviviruses are primarily neurotropic (such as West Nile virus), others are primarily viscerotropic (e.g., yellow fever virus and dengue virus), and still others exhibit both properties (such as Kyasanur Forest disease virus). However, both neurotropism and viscerotropism are present to some degree in all flaviviruses. Within the host, an interaction between viscerotropism and neurotropism is likely to occur, because infection of viscera occurs before invasion of the central nervous system. Thus, neurotropism depends on the ability of the virus to replicate in extraneural organs (viscera). This extraneural replication produces viremia, which in turn is responsible for invasion of the brain and spinal cord.

One approach to developing vaccines against flaviviruses is to modify their virulence properties, so that the vaccine virus has lost its neurotropism and viscerotropism for humans or animals. In the case of yellow fever virus, two vaccines (yellow fever 17D and the French neurotropic vaccine) have been developed (Monath, “Yellow Fever,” In Plotkin and Orenstein, Vaccines, 3^{rd} ed., 1999, Saunders, Philadelphia, pp. 815-879). The yellow fever 17D vaccine was developed by serial passage in chicken embryo tissue, and resulted in a virus with significantly reduced neurotropism and viscerotropism. The French neurotropic vaccine was developed by serial passages of the virus in mouse brain tissue, and resulted in loss of viscerotropism, but retained neurotropism. A high incidence of neurological accidents (post-vaccinal encephalitis) was associated with the use of the French vaccine. Approved vaccines are not currently available for many medically important flaviviruses having viscerotropic properties, such as dengue, West Nile, and Omsk hemorrhagic fever viruses, among others.

Fully processed, mature virions of flaviviruses contain three structural proteins, capsid (C), membrane (M), and envelope (E). Seven non-structural proteins (NS 1, NS2a, NS2b, NS3, NS4a, NS4b, and NS5) are produced in infected cells. Both viral receptor and fusion domains reside within the E protein. Further, the E protein is also a desirable component of flavivirus vaccines, since antibodies against this protein can neutralize virus infectivity and confer protection on the host against the disease. Immature flavivirions found in infected cells contain pre-membrane (prM) protein, which is a precursor to the M protein. The flavivirus proteins are produced by translation of a single, long open reading frame to generate a polyprotein, followed by a complex series of post-translational proteolytic cleavages of the polyprotein, to generate mature viral proteins (Amberg et al., J. Virol. 73:8083-8094, 1999; Rice, “Flaviviridae,” In Virology, Fields (ed.), Raven-Lippincott, New York, 1995, Volume I, p. 937). The virus structural proteins are arranged in the polyprotein in the order C-prM-E.

### SUMMARY OF THE INVENTION

The invention provides flaviviruses including one or more hinge region (e.g., hydrophobic pocket region) mutations that attenuate the viruses by, e.g., reducing their viscerotropism. These flaviviruses can be, for example, yellow fever virus (e.g., a yellow fever virus vaccine strain); a viscerotropic flavivirus selected from the group consisting of Dengue virus, West Nile virus, Wesselsbron virus, Kyasanur Forest Disease virus, and Omsk Hemorrhagic fever virus; or a chimeric flavivirus. In one example of a chimeric flavivirus, the chimera includes the capsid and non-structural proteins of a first flavivirus virus (e.g., a yellow fever virus) and the pre-membrane and envelope proteins of a second flavivirus (e.g., a Japanese encephalitis virus or a Dengue virus (e.g., Dengue virus 1, 2, 3, or 4)) including an envelope protein mutation that decreases viscerotropism of the chimeric flavivirus. In the case of Dengue virus, the mutation can be, for example, in the lysine at Dengue envelope amino acid position 202 (dengue 3) or 204 (dengue 1, 2, and 4). This amino acid can be substituted by, for example, arginine.

The invention also provides vaccine compositions that include any of the viruses described herein and a pharmaceutically acceptable carrier or diluent, as well as methods of inducing an immune response to a flavivirus in a patient by administration of such a vaccine composition to the patient. Patients treated using these methods may not have, but be at risk of developing, the flavivirus infection, or may have the flavivirus infection.

Also included in the invention are methods of producing flavivirus vaccines, involving introducing into a flavivirus (e.g., a chimeric flavivirus) a mutation that results in decreased viscerotropism. Further, the invention includes methods of identifying flavivirus (e.g., yellow fever virus or chimeric flavivirus) vaccine candidates, involving (i) introducing a mutation into the hinge region (e.g., the hydrophobic pocket region) of a flavivirus; and (ii) determining whether the flavivirus including the mutation has decreased viscerotropism, as compared with a flavivirus virus lacking the mutation.

Flaviviruses of the invention are advantageous because, in having decreased viscerotropism, they provide an additional level of safety, as compared to their non-mutated counterparts, when administered to patients. Additional advantages of these viruses are provided by the fact that they can include sequences of yellow fever virus strain YF17D (e.g., sequences encoding capsid and non-structural proteins), which (i) has had its safety established for >60 years, during which over 350 million doses have been administered to humans, (ii) induces a long duration of immunity after a single dose, and (iii) induces immunity rapidly, within a few days of inoculation. In addition, the vaccine viruses of the invention cause an active infection in the treated patients. As the cytokine milieu and innate immune response of immunized individuals are similar to those in natural infection, the antigenic mass expands in the host, properly folded conformational epitopes are processed efficiently, the adaptive immune response is robust, and memory is established. Moreover, in certain chimeras of the invention, the prM and E proteins derived from the target virus contain the critical antigens for protective humoral and cellular immunity.

Other features and advantages of the invention will be apparent from the following detailed description, the drawings, and the claims.

### BRIEF DESCRIPTION OF THE DRAWINGS

FIG. 1 is a series of graphs showing survival distributions of YF-VAX® and ChimeriVax™-JE constructs, with and without a mutation at E279 (M→K). Four day-old suckling mice inoculated by the intracerebral route with (FIG. 1A) approximately 0.7 log₁₀ PFU, (FIG. 1B) approximately 1.7 log₁₀ PFU, and (FIG. 1C)˜2.7 log₁₀ PFU.

FIG. 2 is a graph of regression analysis, mortality vs. virus dose, showing similar slopes and parallel lines for viruses with (FRhL₅) and without (FRhL₃) the Met to Lys reversion, allowing statistical comparison. The FRhL₅ virus was 18.52 times more potent (virulent) than FRhL₃ (p<0.0001).

FIG. 3 shows the results of independent RNA transfection and passage series of ChimeriVax™-JE virus in FRhL and Vero cells. The emergence of mutations in the prME genes by passage level is shown.

FIG. 4 is a three-dimensional model of the flavivirus envelope glycoprotein ectodomain showing locations of mutations in the hinge region occurring with adaptation in FRhL or Vero cells. The sequence of the JE envelope glycoprotein (strain JaOArS982; Sumiyoshi et al., Virology 161:497-510, 1987) was aligned to one of the TBE structural templates (Rey et al., Nature 375:291-298, 1995) as an input for automated homology modeling building by the method of SegMod (Segment Match Modeling) using LOOK software (Molecular Application Group, Palo Alto, Calif.).

FIG. 5 is a graph showing growth kinetics of ChimeriVax™-DEN1 PMS (wt prME, P7), ChimeriVax™-DEN1 (containing an amino acid substitution from K to R in the envelope protein E (E204 K to R), P10) Vaccine, WT DEN1 PUO359, and YF-VAX® in HepG2 cells. ▪: WT DEN1 (parent PU0359), ♦: ChimeriVax™-DEN1 P7, ▴: ChimeriVax™-DEN1 P10, and ●: YF-VAX.

FIG. 6 is a graph showing growth of virus in IT inoculated Aedes aegypti. Growth of ChimeriVax™-DEN1 PMS ((wt prME, P7), Vaccine (containing an amino acid substitution from K to R in the envelope protein E (E204 K to R), P10), YF-VAX®, and WT DEN1 (strain PUO359, donor of PrME genes for ChimeriVax™-DEN1 virus) viruses in IT-inoculated Aedes aegypti. ▪: WT DEN1 (parent PUO359), ♦: ChimeriVax™-DEN1 P7, ▴: ChimeriVax™-DEN1 P10, and ●: YF-VAX.

FIG. 7 is a three-dimensional model showing the structure of DEN1 E-protein dimer (amino acids 1-394) of ChimeriVax™-DEN1 virus. A: Position of positively charged lysine (K) amino acid at residue 204 of P7 (PMS, 204K) virus is shown by CPK (displays spheres sized to the van der Waals (VDW) radii) representation. Three structural domains are defined by red (domain I), yellow (domain II), and blue (domain III). The structure was built based on the atomic coordinates (1OKE.pdb) of DEN2 virus obtained from protein data bank deposited by Modis et al., Proc. Natl. Acad. Sci. U.S.A. 100(12):6986-6991, 2003, using the homology modeling software (DS modeling 1.1) from Accelrys Inc. (San Diego, Calif.). B: Close up of the marked area in A with K amino acid shown in stick representation. C: The same area as in A from the E-protein model of the mutant DEN1 virus (P10, 204R shown in red). The distances between nitrogen (N) of 204K or 204R and N of 261 H or oxygen (O) of 252V (the opposite strand) are shown in angstrom units. Selected amino acids in B and C are shown in stick representation. Grey, carbon (C); blue, nitrogen (N); red, oxygen (O), and yellow, sulfur (S).

### DETAILED DESCRIPTION

The invention provides flaviviruses (e.g., yellow fever viruses and chimeric flaviviruses) having one or more mutations in the hinge region (e.g., the hydrophobic pocket) of the envelope protein, methods for making such flaviviruses, and methods for using these flaviviruses to prevent or to treat flavivirus infection. The invention is based, in part, on our discovery that viruses having certain mutations in this region are attenuated. For example, we have found that viruses having hinge region mutations have decreased viscerotropism (see below). The viruses and methods of the invention are described further, as follows.

One example of a flavivirus that can be used in the invention is yellow fever virus. Mutations can be made in the hinge region of the envelope of a wild-type infectious clone, e.g., the Asibi infectious clone or an infectious clone of another wild-type, virulent yellow fever virus, and the mutants can then be tested in an animal model system (e.g., in hamster and/or monkey model systems) to identify sites affecting viscerotropism. Reduction in viscerotropism is judged by, for example, detection of decreased viremia and/or liver injury in the model system (see below for additional details). One or more mutations found to decrease viscerotropism of the wild-type virus are then introduced into a vaccine strain (e.g., YF17D), and these mutants are tested in an animal model system (e.g., in a hamster and/or a monkey model system) to determine whether the resulting mutants have decreased viscerotropism. Mutants that are found to have decreased viscerotropism can then be used as new vaccine strains that have increased safety, due to decreased levels of viscerotropism.

Additional flaviviruses that can be used in the invention include other mosquito-borne flaviviruses, such as Japanese encephalitis, Dengue (serotypes 1-4), Murray Valley encephalitis, St. Louis encephalitis, West Nile, Kunjin, Rocio encephalitis, and Ilheus viruses; tick-borne flaviviruses, such as Central European encephalitis, Siberian encephalitis, Russian Spring-Summer encephalitis, Kyasanur Forest Disease, Omsk Hemorrhagic fever, Louping ill, Powassan, Negishi, Absettarov, Hansalova, Apoi, and Hypr viruses; as well as viruses from the Hepacivirus genus (e.g., Hepatitis C virus). All of these viruses have some propensity to infect visceral organs. The viscerotropism of these viruses may not cause dysfunction of vital visceral organs, but the replication of virus in these organs can cause viremia and thus contribute to invasion of the central nervous system. Decreasing the viscerotropism of these viruses by mutagenesis can thus reduce their abilities to invade the brain and cause encephalitis.

In addition to the viruses listed above, as well as other flaviviruses, chimeric flaviviruses that include one or more mutations in the envelope protein hinge region (e.g., the hydrophobic pocket) are included in the invention. These chimeras can consist of a flavivirus (i.e., a backbone flavivirus) in which a structural protein (or proteins) has been replaced with a corresponding structural protein (or proteins) of a second virus (i.e., a test or a predetermined virus, such as a flavivirus). For example, the chimeras can consist of a backbone flavivirus (e.g., a yellow fever virus) in which the prM and E proteins of the flavivirus have been replaced with the prM and E proteins of the second, test virus (e.g., a dengue virus (1-4), Japanese encephalitis virus, West Nile virus, or another virus, such as any of those mentioned herein), the E protein of which has a hinge region mutation as described herein. The chimeric viruses can be made from any combination of viruses. Preferably, the virus against which immunity is sought is the source of the inserted structural protein(s).

A specific example of a chimeric virus that can be included in the vaccines of the invention is the yellow fever human vaccine strain, YF17D, in which the prM protein and the E protein have been replaced with the prM protein and the E protein (including a hinge mutation as described herein) of another flavivirus, such as a Dengue virus (serotype 1, 2, 3, or 4), Japanese encephalitis virus, West Nile virus, St. Louis encephalitis virus, Murray Valley encephalitis virus, or any other flavivirus, such as one of those listed above. For example, the following chimeric flaviviruses, which were deposited with the American Type Culture Collection (ATCC) in Manassas, Va., U.S.A. under the terms of the Budapest Treaty and granted a deposit date of Jan. 6, 1998, can be used to make viruses of the invention: Chimeric Yellow Fever 17D/Dengue Type 2 Virus (YF/DEN-2; ATCC accession number ATCC VR-2593) and Chimeric Yellow Fever 17D/Japanese Encephalitis SA14-14-2 Virus (YF/JE A1.3; ATCC accession number ATCC VR-2594). Details of making chimeric viruses that can be used in the invention are provided, for example, in International applications PCT/US98/03894 and PCT/US00/32821, and in Chambers et al., J. Virol. 73:3095-3101, 1999, each of which is incorporated by reference herein in its entirety.

As is noted above, mutations that are included in the viruses of the present invention attenuate the viruses by, e.g., decreasing their viscerotropism. These mutations can be present in the hinge region of the flavivirus envelope protein. The polypeptide chain of the envelope protein folds into three distinct domains: a central domain (domain I), a dimerization domain (domain II), and an immunoglobulin-like module domain (domain III). The hinge region is present between domains I and II and, upon exposure to acidic pH, undergoes a conformational change (hence the designation “hinge”) that results in the formation of envelope protein trimers that are involved in the fusion of viral and endosomal membranes, after virus uptake by receptor-mediated endocytosis. Prior to the conformational change, the proteins are present in the form of dimers.

Numerous envelope amino acids are present in the hinge region including, for example, amino acids 48-61, 127-131, and 196-283 of yellow fever virus (Rey et al., Nature 375:291-298, 1995). Any of these amino acids, or closely surrounding amino acids (and corresponding amino acids in other flavivirus envelope proteins), can be mutated according to the invention, and tested for attenuation. Of particular interest are amino acids within the hydrophobic pocket of the hinge region. As a specific example, and is described further below, we have found that substituting envelope protein amino acid 204 (K to R), which is in the hydrophobic pocket of the hinge region, in a chimeric flavivirus including dengue 1 sequences inserted into a yellow fever virus vector results in attenuation. Also described below is our discovery that this substitution leads to an alteration in the structure of the envelope protein, such that intermolecular hydrogen bonding between one envelope monomer and another in the wild type protein is disrupted and replaced with new intramolecular interactions within monomers. We propose that the attenuation resulting from this substitution is due to these new interactions, which change the structure of the protein in the pre-fusion conformation, most likely by alterating the pH threshold that is required for fusion of viral membrane with the host cell. This discovery thus provides a new basis for the design of further attenuated mutants. In particular, additional substitutions can be used to increase intramolecular interactions in the hydrophobic pocket, leading to attenuation. Examples of such mutations/substitutions that can be made in the hydrophobic pocket, according to the invention, include substitutions in E202K, E204K, E252V, E253L, E257E E258G, and E261H.

Mutations can be made in the hinge region using standard methods, such as site-directed mutagenesis. One example of the type of mutation present in the viruses of the invention is substitutions, but other types of mutations, such as deletions and insertions, can be used as well. In addition, as is noted above, the mutations can be present singly or in the context of one or more additional mutations.

The viruses (including chimeras) of the present invention can be made using standard methods in the art. For example, an RNA molecule corresponding to the genome of a virus can be introduced into primary cells, chick embryos, or diploid cell lines, from which (or the supernatants of which) progeny virus can then be purified. Another method that can be used to produce the viruses employs heteroploid cells, such as Vero cells (Yasumura et al., Nihon Rinsho 21, 1201-1215, 1963). In this method, a nucleic acid molecule (e.g., an RNA molecule) corresponding to the genome of a virus is introduced into the heteroploid cells, virus is harvested from the medium in which the cells have been cultured, harvested virus is treated with a nuclease (e.g., an endonuclease that degrades both DNA and RNA, such as Benzonase™; U.S. Pat. No. 5,173,418), the nuclease-treated virus is concentrated (e.g., by use of ultrafiltration using a filter having a molecular weight cut-off of, e.g., 500 kDa), and the concentrated virus is formulated for the purposes of vaccination. Details of this method are provided in U.S. Patent Application Ser. No. 60/348,565, filed Jan. 15, 2002, which is incorporated herein by reference (also see WO 03/060088 A2).

The viruses of the invention can be administered as primary prophylactic agents in adults or children at risk of infection, or can be used as secondary agents for treating infected patients. Formulation of the viruses of the invention can be carried out using methods that are standard in the art. Numerous pharmaceutically acceptable solutions for use in vaccine preparation are well known and can readily be adapted for use in the present invention by those of skill in this art (see, e.g., Remington's Pharmaceutical Sciences (18^{th} edition), ed. A. Gennaro, 1990, Mack Publishing Co., Easton, Pa.). In two specific examples, the viruses are formulated in Minimum Essential Medium Earle's Salt (MEME) containing 7.5% lactose and 2.5% human serum albumin or MEME containing 10% sorbitol. However, the viruses can simply be diluted in a physiologically acceptable solution, such as sterile saline or sterile buffered saline. In another example, the viruses can be administered and formulated, for example, in the same manner as the yellow fever 17D vaccine, e.g., as a clarified suspension of infected chicken embryo tissue, or a fluid harvested from cell cultures infected with the chimeric yellow fever virus.

The vaccines of the invention can be administered using methods that are well known in the art, and appropriate amounts of the vaccines administered can be readily be determined by those of skill in the art. For example, the viruses of the invention can be formulated as sterile aqueous solutions containing between 10² and 10⁷ infectious units (e.g., plaque-forming units or tissue culture infectious doses) in a dose volume of 0.1 to 1.0 ml, to be administered by, for example, intramuscular, subcutaneous, or intradermal routes. In addition, because flaviviruses may be capable of infecting the human host via the mucosal routes, such as the oral route (Gresikova et al., “Tick-borne Encephalitis,” In The Arboviruses, Ecology and Epidemiology, Monath (ed.), CRC Press, Boca Raton, Fla., 1988, Volume IV, 177-203), the viruses can be administered by mucosal routes as well. Further, the vaccines of the invention can be administered in a single dose or, optionally, administration can involve the use of a priming dose followed by a booster dose that is administered, e.g., 2-6 months later, as determined to be appropriate by those of skill in the art.

Optionally, adjuvants that are known to those skilled in the art can be used in the administration of the viruses of the invention. Adjuvants that can be used to enhance the immunogenicity of the viruses include, for example, liposomal formulations, synthetic adjuvants, such as (e.g., QS21), muramyl dipeptide, monophosphoryl lipid A, or polyphosphazine. Although these adjuvants are typically used to enhance immune responses to inactivated vaccines, they can also be used with live vaccines. In the case of a virus delivered via a mucosal route, for example, orally, mucosal adjuvants such as the heat-labile toxin of E. coli (LT) or mutant derivations of LT can be used as adjuvants. In addition, genes encoding cytokines that have adjuvant activities can be inserted into the viruses. Thus, genes encoding cytokines, such as GM-CSF, IL-2, IL-12, IL-13, or IL-5, can be inserted together with foreign antigen genes to produce a vaccine that results in enhanced immune responses, or to modulate immunity directed more specifically towards cellular, humoral, or mucosal responses.

In the case of dengue virus, against which optimal vaccination can involve the induction of immunity against all four of the dengue serotypes, the chimeric viruses of the present invention can be used in the formulation of tetravalent vaccines. Any or all of the chimeras used in such tetravalent formulations can include a mutation that decreases viscerotropism, as is described herein. The chimeras can be mixed to form tetravalent preparations at any point during formulation, or can be administered in series. In the case of a tetravalent vaccine, equivalent amounts of each chimera may be used. Alternatively, the amounts of each of the different chimeras present in the administered vaccines can vary. Briefly, in one example of such a formulation, at least 5 fold less of the dengue-2 chimera (e.g., 10, 50, 100, 200, or 500 fold less) is used relative to the other chimeras. In this example, the amounts of the dengue-1, dengue-3, and dengue-4 chimeras can be equivalent or can vary. In another example, the amounts of dengue-4 and/or dengue 1 virus can be decreased as well. For example, in addition to using less dengue-2 chimera, at least 5 fold less of the dengue-4 chimera (e.g., 10, 50, 100, 200, or 500 fold less) can be used relative to the dengue-1 and dengue-3 chimeras; at least 5 fold less of the dengue-l chimera (e.g., 10, 50, 100, 200, or 500 fold less) can be used relative to the dengue-3 and dengue-4 chimeras; or at least 5 fold less of the dengue- 1 and dengue-4 chimeras can be used relative to the dengue-3 chimera. It may be particularly desirable, for example, to decrease the amount of dengue-1 chimera relative to the amounts of dengue-3 and/or dengue-4 chimeras when the E204/E202 mutation described herein is not included in the chimera.

Details of the characterization of one example of a mutation included in the invention, which occurs at position 279 of the envelope protein of a yellow fever/Japanese encephalitis chimera, are provided below. Also provided below are details concerning yellow fever/dengue virus chimeras, in which dengue virus envelope proteins include one or more mutations that decrease viscerotropism. In one example of such a mutation, the lysine at position 204 of the envelope protein of dengue-1, dengue-2, or dengue-4, or the lysine at position 202 of the envelope protein of dengue-3, which is two amino acids shorter than the envelope proteins of the other dengue serotypes, is substituted or deleted. This lysine can be, for example, substituted with arginine. Other residues near envelope amino acid 204 (202 for dengue-3) can also be mutated to achieve decreased viscerotropism. For example, any of amino acids 200-208 or combinations of these amino acids can be mutated. Specific examples include the following: position 202 (K) and 204 (K) of dengue-1, dengue 2, and dengue 4 and position 200 (K) and 202 (K) of dengue 3. These residues can be substituted with, for example, arginine.

Experimental Results

I. Yellow Fever/Japanese Encephalitis Chimera Including a Hinge Region Mutation

Summary

A chimeric yellow fever (YF)-Japanese encephalitis (JE) vaccine (ChimeriVax™-JE) was constructed by insertion of the prM-E genes from the attenuated JE SA14-14-2 vaccine strain into a full-length cDNA clone of YF 17D virus. Passage in fetal rhesus lung (FRhL) cells led to the emergence of a small-plaque virus containing a single Met→Lys amino acid mutation at E279, reverting this residue from the SA14-14-2 to the wild-type amino acid. A similar virus was also constructed by site-directed mutagenesis. The E279 mutation is located in a beta-sheet in the hinge region of the E protein, which is responsible for a pH-dependent conformational change during virus penetration from the endosome into the cytoplasm of an infected cell. In independent transfection-passage studies in FRhL or Vero cells, mutations appeared most frequently in hinge 4 (bounded by amino acids E266 to E284), reflecting genomic instability in this functionally important region. The E279 reversion caused a significant increase in neurovirulence, as determined by LD50 and survival distribution in suckling mice and by histopathology in rhesus monkeys. Based on sensitivity and comparability of results with monkeys, the suckling mouse is an appropriate host for safety testing of flavivirus vaccine candidates for neurotropism. The E279 Lys virus was restricted with respect to extraneural replication in monkeys, as viremia and antibody levels (markers of viscerotropism) were significantly reduced as compared to E279 Met virus.

Background

The study of chimeric viruses has afforded new insights into the molecular basis of virulence and new prospects for vaccine development. For example, molecular clones of positive-strand alphaviruses (Morris-Downes et al., Vaccine 19:3877-3884, 2001; Xiong et al., Science 243:1188-1191, 1991) and flaviviruses (Bray et al., Proc. Natl. Acad. Sci. U.S.A. 88:10342-10346, 1991; Chambers et al., J. Virol. 73:3095-3101, 1999; Guirakhoo et al., J. Virol. 75:7290-7304, 2001; Huang et al., J. Virol. 74:3020-3028, 2000) have been modified by insertion of structural genes encoding the viral envelope and determinants involved in neutralization, cell attachment, fusion, and internalization. The replication of these chimeric viruses is controlled by nonstructural proteins and the non-coding termini expressed by the parental strain, while the structural proteins from the donor genes afford specific immunity. The biological characteristics of chimeric viruses are determined by both the donor and recipient virus genes. By comparing constructs with nucleotide sequence differences across the donor genes, it is possible to dissect out the functional roles of individual amino acid residues in virulence and attenuation.

Using a chimeric yellow fever (YF) virus that incorporated the prM-E genes from an attenuated strain (SA14-14-2) of Japanese encephalitis (JE), a detailed examination was made of the role of 10 amino acid mutations that distinguished the attenuated JE virus from virulent, wild-type JE Nakayama virus (Arroyo et al., J. Virol. 75:934-942, 2001). The virulence factors were defined by reverting each mutation singly or as clusters to the wild-type sequence and determining the effects on neurovirulence for young adult mice inoculated by the intracerebral (IC) route with 10⁴ plaque-forming units (PFU). All of the single-site revertant viruses remained highly attenuated, and reversions at 3 or 4 residues were required to restore a neurovirulent phenotype. Only one single-site revertant (E279 Met→Lys) showed any evidence of a change in virulence, with 1 of 8 animals succumbing after IC inoculation.

In order to explore further the functional role of the E279 determinant, we compared chimeric YF/JE viruses that differed at this amino acid residue for their abilities to cause encephalitis in suckling mice and monkeys. IC inoculation of monkeys is routinely used as a test for safety of flavivirus and other live vaccines, and quantitative pathological examination of brain and spinal cord tissue provides a sensitive method for distinguishing strains of the same virus with subtle differences in neurovirulence (Levenbook et al., J. Biol. Stand. 15: 305-313, 1987). Suckling mice provide a more sensitive model than older animals, since susceptibility to neurotropic flaviviruses is age-dependent (Monath et al., J. Virol. 74:1742-1751, 2000). The results confirmed that the single Met→Lys amino acid mutation at E279 conferred an increase in neurovirulence. This mutation is located in the ‘hinge’ region of the E protein, which is responsible for a pH-dependent conformational change during virus penetration from the endosome into the cytoplasm of an infected cell (Reed et al., Am. J. Hyg. 27:493-497, 1938). Importantly, the suckling mouse was shown to predict the virulence profile in rhesus monkeys. Based on the detection of a change in neurovirulence conferred by a point mutation, we propose that the suckling mouse is an appropriate host for safety testing of flavivirus vaccine candidates for neurotropism.

While enhancing neurovirulence, the E279 mutation appeared to have the opposite effect on viscerotropism, as measured by decreased viremia and antibody response in monkeys, accepted markers of this viral trait (Wang et al., J. Gen. Virol. 76:2749-2755, 1995).

Materials and Methods

Viruses

Development of the ChimeriVax™-JE vaccine began by cloning a cDNA copy of the entire 11-kilobase genome of YF 17D virus (Chambers et al., J. Virol. 73:3095-3101, 1999). To accomplish this, YF 17D genomic sequences were propagated in two plasmids, which encode the YF sequences from nucleotide (nt) 1-2276 and 8279-10,861 (plasmid YF5′3′IV), and from 1373-8704 (plasmid YFM5.2), respectively. Full-length cDNA templates were generated by ligation of appropriate restriction fragments derived from these plasmids. YF sequences within the YF 5′3′IV and YFM5.2 plasmids were replaced by the corresponding JE (SA14-14-2) pr-ME sequences, resulting in the generation of YF5′3′IV/JE (prM-E′) and YFM5.2/JE (E′-E) plasmids. These plasmids were digested sequentially with restriction endonucleases NheI and BspEI. Appropriate fragments were ligated with T4 DNA ligase, cDNA was digested with XhoI enzyme to allow transcription, and RNA was produced from an Sp6 promoter. Transfection of diploid fetal rhesus lung (FRhL) cells with full-length RNA was performed by electroporation. Supernatant containing virus was harvested when cytopathic effect was observed (generally day 3), clarified by low-speed centrifugation and sterile-filtered at 0.22 μm. Fetal bovine serum (FBS) 50% v/v final concentration was added as a stabilizer. The virus was titrated by plaque assay in Vero cells, as previously described (Monath et al., Vaccine 17:1869-1882, 1999). The chimeric virus was sequentially passed in FRhL or Vero cells (Vero-PM, Aventis Pasteur, Marcy l'Étoile, France) at a multiplicity of infection of approximately 0.001. Commercial yellow fever 17D vaccine (YF-VAX®) was obtained from Aventis-Pasteur (formerly Pasteur-Merieux-Connaught), Swiftwater, Pa.

Site-Directed Mutagenesis

Virus containing a single-site Met→Lys reversion at residue E279 was generated by oligo-directed mutagenesis as described (Arroyo et al., J. Virol. 75:934-942, 2001). Briefly, a plasmid (pBS/JE SA14-14-2) containing the JE SA-14-14-2 E gene region from nucleotides 1108 to 2472 (Cecilia et al., Virology 181:70-77, 1991) was used as template for site-directed mutagenesis. Mutagenesis was performed using the Transformer site-directed mutagenesis kit (Clontech, Palo Alto, Calif.) and oligonucleotide primers synthesized at Life Technologies (Grand Island, N.Y.). Plasmids were sequenced across the E region to verify that the only change was the engineered mutation. A region encompassing the E279 mutation was then subcloned from the pBS/JE plasmid into pYFM5.2/JE SA14-14-2 (Cecilia et al., Virology 181:70-77, 1991) using the NheI and EheI (Kas I) restriction sites. Assembly of full-length DNA and SP6 transcription were performed as described above; however, RNA transfection of Vero cells was performed using Lipofectin (Gibco/BRL).

Sequencing

RNA was isolated from infected monolayers by Trizol® (Life Technologies). Reverse transcription was performed with Superscript II Reverse Transcriptase (RT) and a long-RT protocol (Life Technologies), followed by RNaseH treatment (Promega) and long-PCR (XL PCR, Perkin-Elmer/ABI). RT, PCR, and sequencing primers were designed using YF17D strain sequence (GeneBank Accession number K02749) and JE-SA14-14-2 strain sequence (GeneBank Accession number D90195) as references. PCR products were gel-purified (Qiaquick gel-extraction kit from Qiagen) and sequenced using Dye-Terminator dRhodamine sequencing reaction mix (Perkin-Elmer/ABI). Sequencing reactions were analyzed on a model 310 Genetic Analyzer (Perkin-Elmer/ABI) and DNA sequences were evaluated using Sequencher 3.0 (GeneCodes) software.

Plaque Assays and Neutralization Tests

Plaque assays were performed in 6 well plates of monolayer cultures of Vero cells. After adsorption of virus during a 1 hour incubation at 37° C., the cells were overlaid with agarose in nutrient medium. On day 4, a second overlay was added containing 3% neutral red. Serum-dilution, plaque-reduction neutralization tests were performed as previously described (Monath et al., Vaccine 17:1869-1882, 1999).

Weaned Mouse Model

Groups of 8 to 10 female 4 week old ICR mice (Taconic Farms, Inc. Germantown, N.Y.) were inoculated IC with 30 μL of chimeric YF/JE SA14-14-2 (ChimeriVax™-JE) constructs with (dose 4.0 log₁₀ PFU in) or without (3.1 log₁₀ PFU) the E279 mutation. An equal number of mice were inoculated with YF-VAX® or diluent. Mice were followed for illness and death for 21 days.

Suckling Mouse Model

Pregnant female ICR mice (Taconic Farms) were observed through parturition in order to obtain litters of suckling mice of exact age. Suckling mice from multiple litters born within a 48 hour interval were pooled and randomly redistributed to mothers in groups of up to 121 mice. Litters were inoculated IC with 20 μL of serial tenfold dilutions of virus and followed for signs of illness and death for 21 days. The virus inocula were back-titrated. 50% lethal dose (LD₅₀) values were calculated by the method of Reed and Muench (Morris-Downes et al., Vaccine 19:3877-3884, 2001). Univariate survival distributions were plotted and compared by log rank test.

Monkey Model

The monkey neurovirulence test was performed as described by Levenbook et al. (Levenbook et al., J. Biol. Stand. 15: 305-313, 1987) and proscribed by WHO regulations for safety testing YF 17D seed viruses (Wang et al., J. Gen. Virol. 76:2749-2755, 1995). This test has previously been applied to the evaluation of ChimeriVax™-JE vaccines, and results of tests on FRhL₃ virus were described (Monath et al., Curr. Drugs- Infect. Dis., 1:37-50; 2001; Monath et al., Vaccine 17:1869-1882, 1999). Tests were performed at Sierra Biomedical Inc. (Sparks, Nev.), according to the U.S. Food and Drug Administration Good Laboratory Practice (GLP) regulations (21 C.F.R., Part 58). On Day 1, ten (5 male, 5 female) rhesus monkeys weighing 3.0-6.5 kg received a single inoculation of 0.25 mL undiluted ChimeriVax™-JE virus with or without the E279 Met→Lys mutation or YF-VAX® into the frontal lobe of the brain. Monkeys were evaluated daily for clinical signs and scored as 0 (no signs), 1 (rough coat, not eating), 2 (high-pitched voice, inactive, slow moving, 3 (shaky movements, tremors, incoordination, limb weakness), and 4 (inability to stand, limb paralysis, death). The clinical score for each monkey is the mean of the animal's daily scores, and the clinical score for the treatment group is the arithmetic mean of the individual clinical scores. Viremia levels were measured by plaque assay in Vero cells using sera collected on days 2-10. On day 31, animals were euthanized, perfused with isotonic saline-5%acetic acid followed by neutral-buffered 10% formalin, and necropsies were performed. Brains and spinal cords were fixed, sectioned and stained with gallocyanin. Neurovirulence was assessed by the presence and severity of lesions in various anatomical formations of the central nervous system. Severity was scored within each tissue block using the scale specified by WHO (Wang et al., J. Gen. Virol. 76:2749-2755, 1995):
Grade 1: Minimal: 1-3 small focal inflammatory infiltrates. A few neurons may be changed or lost.
Grade 2: Moderate: more extensive focal inflammatory infiltrates. Neuronal changes or loss affects not more than one-third of neurons.
Grade 3: Severe: neuronal changes or loss affecting 33-90% of neurons; moderate focal or diffuse inflammatory changes
Grade 4: Overwhelming; more than 90% of neurons are changed or lost, with variable but frequently severe inflammatory infiltration

Structures involved in the pathologic process most often and with greatest severity were designated ‘target areas,’ while those structures discriminating between wild-type JE virus and ChimeriVax™-JE were designated ‘discriminator areas.’ The substantia nigra constituted the ‘target area’ and the caudate nucleus, globus pallidus, putamen, anterior/medial thalamic nucleus, lateral thalamic nucleus, and spinal cord (cervical and lumbar enlargements) constituted ‘discriminator areas’ (Monath et al., Curr. Drugs Infect. Dis., 1:37-50, 2001), as previously shown for YF 17D (Levenbook et al., J. Biol. Stand. 15:305-313, 1987). All neuropathological evaluations were done by a single, experienced investigator who was blinded to the treatment code. Separate scores for target area, discriminator areas, and target+discriminator areas were determined for each monkey, and test groups compared with respect to average scores. Other areas of the brainstem (nuclei of the midbrain in addition to substantia nigra; pons; medulla; and cerebellum) and the leptomeninges were also examined. Statistical comparisons of mean neuropathological scores (for the target area, discriminator areas, and target+discriminator areas) were performed by Student's t test, 2-tailed. In addition to neuropathological examination, the liver, spleen, adrenal glands, heart, and kidneys were examined for pathologic changes by light microscopy.

Genome Stability

To ascertain the genetic stability of the YF/JE chimeric virus, and to search for ‘hot spots’ in the vaccine genome that are susceptible to mutation, multiple experiments were performed in which RNA was used to transfect cells and the progeny virus serially passaged in vitro, with partial or complete genomic sequencing performed at low and high passage levels. Passage series were performed starting with the transfection step in FRhL or Vero-PM cells. Serial passage of the virus was performed at low MOI in cell cultures grown in T25 or T75 flasks. At selected passage levels, duplicate samples of viral genomic RNA were extracted, reverse-transcribed, amplified by PCR, and the prM-E region or full genomic sequence determined.

Results

Generation of Single-Site Mutant Viruses by Empirical Passage

The chimeric YF/JESA 14-14-2 (ChimeriVax™-JE) virus recovered from transfected FRhL cells (FRhL₁) was passed sequentially in fluid cultures of these cells at an MOI of approximately 0.001. As is described below, at passage 4 we noted a change in plaque morphology, which was subsequently shown to be associated with a T→G transversion at nucleotide 1818 resulting in an amino acid change (Met→Lys) at position 279 of the E protein.

Plaques were characterized at each passage level and classified into 3 categories based on their sizes measured on day 6 (large, L˜>1.0 mm, medium, M˜0.5-1 mm, and small, S˜<0.5 mm). The plaque size distribution was determined by counting 100 plaques. FRhL₃ (3^{rd} passage) virus contained 80-94% L and 6-20% S plaques. At FRhL₅ (5^{th} passage), a change in plaque size was detected, with the emergence of S plaques comprising >85% of the total plaque population. The FRhL₄ virus was intermediate, with 40% large and 60% small plaques. Full genomic sequencing of the FRhL₅ virus demonstrated a single mutation at E279. The full genome consensus sequence of the FRhL₅ chimera, with careful inspection for codon heterogeneity, confirmed that this was the only detectable mutation present in the virus. The full genome consensus sequence of the FRhL₃ virus revealed no detectable mutations compared to the parental YF/JESA14-14-2 chimeric virus (Arroyo et al., J. Virol. 75:934-942, 2001) (Table 1).

Ten large, medium, and small plaques were picked from FRhL₃, ₋₄ and ₋₅, and amplified by passage in fluid cultures of FRhL cells. After amplification, the supernatant fluid was plaqued on Vero cells. Attempts to isolate the S plaque phenotype from FRhL₃ failed and all isolated L or S size plaques produced a majority of L plaques after one round of amplification in FRhL cells. At the next passage (FRhL₄), where 60% of plaques were of small size, it was possible to isolate these plaques by amplification in FRhL cells. At FRhL , the majority of plaques (85-99%) were of small size, and amplification of both L and S individual plaques resulted in majority of S size. Sequencing the prM-E genes of the S and L plaque phenotypes from FRhL₃ revealed identical sequences to the parent SA14-14-2 genes used for construction of ChimeriVax™-JE, whereas S plaques isolated from either FRhL₄ or FRhL₅ virus revealed the mutation (Met→Lys) at E279.

Animal Protocols

All studies involving mice and nonhuman primates were conducted in accordance with the USDA Animal Welfare Act (9 C.F.R., Parts 1-3) as described in the Guide for Care and Use of Laboratory Animals.

Virulence for Weaned Mice

Ten female ICR mice 4 weeks of age were inoculated IC with approximately 3.0 log₁₀ PFU of FRhL₃, ₋₄, or ₋₅ virus in separate experiments; in each study 10 mice received an equivalent dose (approximately 3.3 log₁₀ PFU) of commercial yellow fever vaccine (YF-VAX®, Aventis Pasteur, Swiftwater Pa.). None of the mice inoculated with chimeric viruses showed signs of illness or died, whereas 70-100% of control mice inoculated with YF-VAX® developed paralysis or died. In another experiment, 8 mice were inoculated IC with FRhL₅ (3.1 log₁₀ PFU) or the YF/JE single-site E279 revertant (4.0 log₁₀ PFU) and 9 mice received YF-VAX® (2.3 log₁₀ PFU). None of the mice inoculated with the chimeric constructs became ill, whereas 6/9 (67%) of mice inoculated with YF-VAX® died.

Virulence for Suckling Mice

Two separate experiments were performed in which YF/JESA14-14-2 chimeric viruses with and without the E279 mutation were inoculated IC at graded doses into suckling mice (Table 2). YF-VAX® was used as the reference control in these experiments. LD₅₀ and average survival times (AST) were determined for each virus.

In the first experiment using mice 8.6 days old, FRhL₅ virus containing the single site reversion (Met→Lys) at E279 was neurovirulent, with a log₁₀ LD₅₀ of 1.64 whereas the FRhL₃ virus lacking this mutation was nearly avirulent, with only 1 of 10 mice dying in the highest dose groups (Table 2). At the highest dose (approximately 3 log₁₀ PFU), the AST of the FRhL₅ virus was shorter (10.3 days) than that of the FRhL₃ virus (15 days).

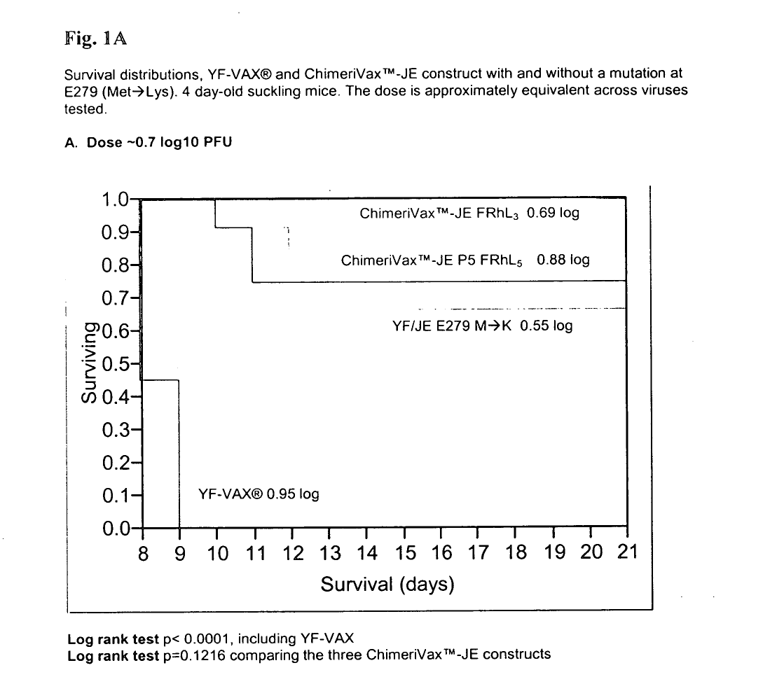

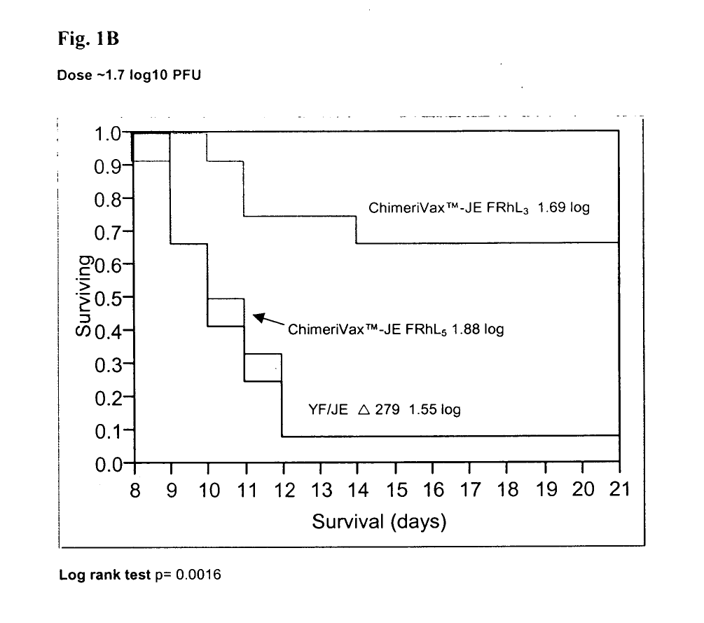

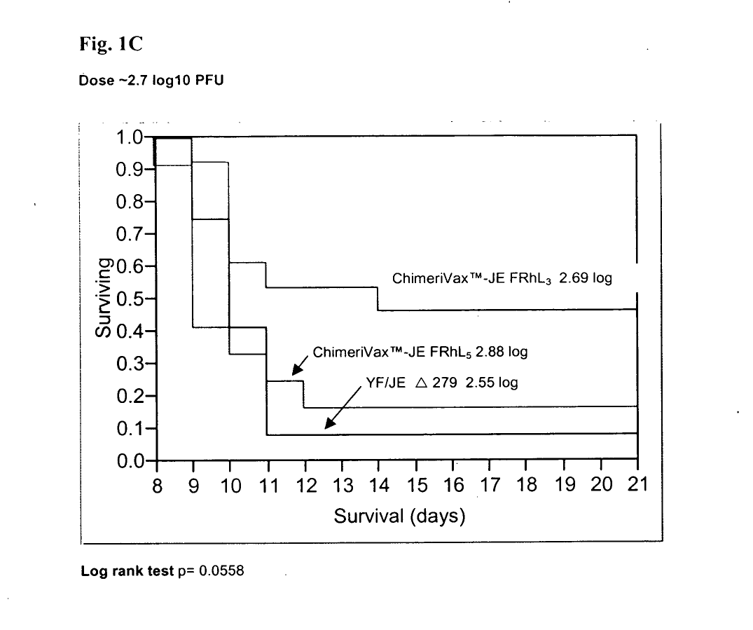

A second experiment was subsequently performed to verify statistically that a single site mutation in the E gene is detectable by neurovirulence test in suckling mice. In this experiment outbred mice 4 days of age were inoculated IC with graded doses of ChimeriVax™-JE FRhL₃ (no mutation), ChimeriVax™-JE FRhL₅ (E279 Met→Lys), or a YF/JE chimera in which a single mutation E279 (Met→Lys) was introduced at by site-directed mutagenesis (Arroyo et al., J. Virol. 75:934-942, 2001). The LD₅₀ values of the two viruses containing the E279 mutation were >10-fold lower than the FRhL₃ construct without the mutation (Table 2) indicating that the E279 Met→Lys mutation increased the neurovirulence of the chimeric virus. There were statistically significant differences between the viruses in the survival distributions (FIG. 1). At the lowest dose (˜0.7 log₁₀ PFU), the YF/JE chimeric viruses were significantly less virulent than YF-VAX® (log rank p<0.0001). The viruses with the E279 Met→Lys mutation had similar survival curves that differed from the FRhL3 virus no mutation), but the difference did not reach statistical significance (log rank p=0.1216). However, at higher doses (˜1.7 and ˜2.7 log₁₀ PFU), the survival distributions of the E279 mutant viruses were significantly different from FRhL₃ virus.

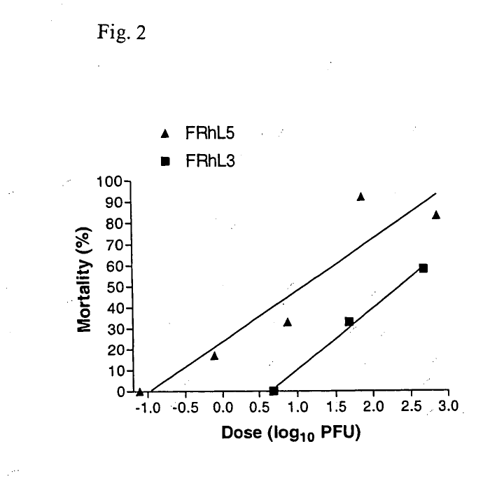

Analysis of mortality ratio by virus dose revealed similar slopes and parallel regression lines (FIG. 2). The FRhL₅ virus was 18.52 times more potent (virulent) than FRhL₃ (95% fiducial limits 3.65 and 124.44, p<0.0001).

Monkey Neurovirulence Test

None of the 20 monkeys inoculated with ChimeriVax™-JE FRhL₃ or FRhL₅ viruses developed signs of encephalitis, whereas 4/10 monkeys inoculated with YF-VAX® developed grade 3 signs (tremors) between days 15-29, which resolved within 6 days of onset. Mean and maximum mean clinical scores were significantly higher in the YF-VAX® group than in the two ChimeriVax™-JE groups. There was no difference in clinical score between groups receiving ChimeriVax™-JE viruses with and without the E279 mutation (Table 3).

There were no differences in weight changes during the experiment between treatment groups. Pathological examination revealed no alterations of liver, spleen, kidney, heart, or adrenal glands attributable to the viruses, and no differences between treatment groups.

Histopathologic examination of the brain and spinal cord revealed significantly higher lesion scores for monkeys inoculated with YF-VAX® than for ChimeriVax™-JE virus FRhL₃ and FRhL₅ (Table 3). The combined target+discriminator scores (±SD) for YF-VAX® was 1.17 (±0.47). The scores for the ChimeriVax™-JE FRhL₃ (E279 Met) and FRhL₅ (E279 Lys) were 0.29 (±0.20), (p=0.00014 vs. YF-VAX®) and 0.54 (±0.28), (p=0.00248 vs. YF-VAX®), respectively.

The discriminator area score and combined target+discriminator area score for ChimeriVax™-JE FRhL₅ containing the Met→Lys reversion at E279 were significantly higher than the corresponding scores for ChimeriVax™-JE FRhL₃ (Table 3).

The main symptom in monkeys inoculated with YF-VAX® was tremor, which may reflect lesions of the cerebellum, thalamic nuclei, or globus pallidus. No clear histological lesions were found in the cerebellar cortex, N. dentatus, or other cerebellar nuclei, whereas imflammatory lesions were present in the thalamic nuclei and globus pallidus in all positive monkeys.

Interestingly, there was an inverse relationship between neurovirulence and viscerotropism of the E279 revertant, as reflected by viremia. The WHO monkey neurovirulence test includes quantitation of viremia as a measure of viscerotropism (World Health Organization, “Requirements for yellow fever vaccine,” Requirements for Biological Substances No. 3, revised 1995, WHO Tech. Rep. Ser. 872, Annex 2, Geneva: WHO, 31-68, 1998). This is rational, based on observations that intracerebral inoculation results in immediate seeding of extraneural tissues (Theiler, “The Virus,” In Strode (ed.), Yellow Fever, McGraw Hill, New York, N.Y., 46-136, 1951). Nine (90%) of 10 monkeys inoculated with YF-VAX® and 8 (80%) of 10 monkeys inoculated with ChimeriVax™-JE FRhL₃ became viremic after IC inoculation. The level of viremia tended to be higher in the YF-VAX® group than in the ChimeriVax™-JE FRhL₃ group, reaching significance on Day 4. In contrast, only 2 (20%) of the animals given FRhL₅ virus (E279 Met→Lys) had detectable, low-level viremias (Table 4), and the mean viremia was significantly lower than FRhL₃ virus on days 3 and 4 (and nearly significant on day 5). Thus, the FRhL₅ revertant virus displayed increased neurovirulence, but decreased viscerotropism compared to FRhL₃ virus. Sera from monkeys inoculated with ChimeriVax™-JE FRhL₃ and FRhL₅ were examined for the presence of plaque size variants. Only L plaques were observed in sera from monkeys inoculated with the FRhL₃, whereas the virus in blood of monkeys inoculated with FRhL₅ had the appropriate S plaque morphology.

Immunogenicity

All monkeys in all three groups developed homologous neutralizing antibodies 31 days post-inoculation to yellow fever (YF-VAX® group) or ChimeriVax™-JE (ChimeriVax™groups), with the exception of 1 animal (FRhL₅, RAK22F), which was not tested due to sample loss. However, the geometric mean antibody titer (GMT) was significantly higher in the monkeys inoculated with FRhL₃ (GMT 501) than with FRhL₅ (GMT 169, p=0.0386, t-test).

Genome Stability

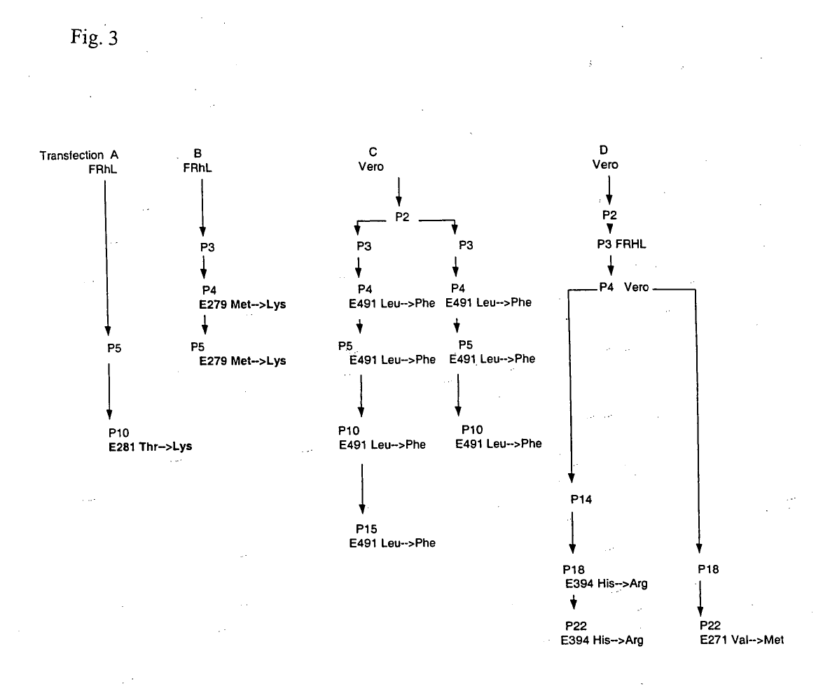

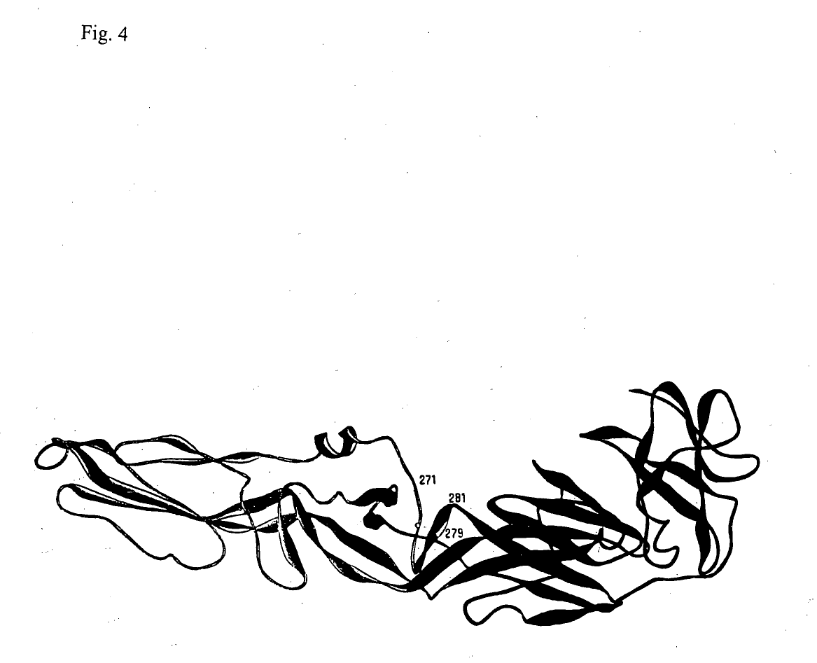

Two separate transfections of ChimeriVax™-JE RNA were performed in each of two cell strains, FRhL and Vero, and progeny viruses were passed as is shown in FIG. 3. The FRhL passage series B resulted in appearance of the E279 reversion at FRhL₄ as described above. Interestingly, a separate passage series (A) in FRhL cells also resulted in the appearance of a mutation (Thr→Lys) in an adjacent residue at E281, and one of the passage series in Vero cells resulted in a Val→Lys mutation at E271. Other mutations selected in Vero cells were in domain III or within the transmembrane domain. All viruses containing mutations shown in FIG. 1 were evaluated in the adult mouse neurovirulence test and were found to be avirulent.

II. Yellow Fever/Dengue Chimera Including a Hinge Region Mutation

Summary

Chimeric yellow fever-denguel virus (ChimeriVax™-DEN1) was produced by transfection of Vero cells with RNA transcribed from chimeric cDNA. The cell culture supernatant was subjected to plaque purification to identify a vaccine candidate without mutations. Out of ten plaque-purified clones, the only one not containing any mutation (clone J) was selected for production of the vaccine virus. However, during cell culture passages of this clone to produce the vaccine, a single amino acid substitution (K to R) occurred at E204. The same mutation was observed in another clone (clone E). This mutation has been found to attenuate the virus for 4 day old suckling mice inoculated by the intracerebral route, and to reduce viremia/viscerotropism in monkeys inoculated by the subcutaneous or intracerebral routes. The clinical scores of lesions in monkey brains inoculated with either virus were statistically lower than that of the control virus, YF-VAX®. Both mutant and parent (non mutant) viruses grew to a significantly lower level than YF-VAX® in HepG2, a human hepatoma cell line. When inoculated into mosquitoes intrathoracically, both viruses grew to a similar level as YF-VAX®, which was significantly lower than that of their wild type DEN1 parent virus. A comparison of the envelope protein structures of parent and mutant viruses revealed the appearance of new intramolecular bonds between 204R, 261 H, and 257E in the mutant virus.

Materials and Methods

Cells and Viruses

Vero cells used for vaccine production were obtained from a qualified cell bank (Aventis Pasteur, France). HepG2 were purchased from American Type Culture Collection (Manassas, Va.). Three-times plaque purified ChimeriVax™-DEN1 viruses (clone E, Vero P6; and clone J, Vero P7) were prepared by transfection of Vero cells with in vitro RNA transcripts and subsequent plaque. ChimeriVax™-DEN1 vaccine lot (VL) virus was produced at P10 from a Pre-Master Seed (PMS; clone J, Vero P7) virus stock by three passages under cGMP manufacture as described. Stock virus of wild type (WT) DEN1 parent (strain PUO359, donor of prME genes for ChimeriVax™-DEN1 virus) was prepared in C6/36 cells. YF-VAX® (vaccine strain 17D) was purchased from Aventis Pasteur (France) and used without any dilutions or further passages. Additional details as to the characterization of various uncloned and cloned ChimeriVax™-DEN1 viruses are provided in Table 5.

Animal Studies

All studies were carried out under an IACUC approved protocol in accordance with the USDA Animal Welfare Act (9 CFR Parts 1-3), as described in the Guide for Care and Use of Laboratory Animals.

Mice

Neurovirulence phenotype of different clones of DEN1 chimeras was assessed in suckling mice. Pregnant ICR mice were purchased from Taconic Farm (Germantown, N.Y.). Suckling mice were pooled at the age of 2-3 days and randomly distributed to mothers (9-12 mice/mother). Mice were inoculated at the age of 3-4 days by the IC route with 0.02 ml of various dilutions of viruses. Mice were observed for 21 days, and mortality recorded. The virus concentrations administered to each group of animals were determined by back titration of inocula in a plaque assay on Vero cells.

Monkeys

Two experiments were performed in macaque monkeys (at Sierra Division, Charles River Laboratories, Inc., Sparks, Nev.) to assess viscerotropism (Experiment 1) and neurovirulence (Experiment 2) of ChimeriVax™-DEN1 viruses with or without the E204 mutation. In the first experiment, rhesus monkeys (Macaca mulatta) were inoculated with chimeric DEN1 viruses by the SC route, whereas in the second experiment cynomolgus monkeys (Macaca fascicularis) were inoculated by the IC route. Because the two species are phylogenetically related and due to unavailability of rhesus monkeys, cynomolgus monkeys were chosen for the second experiment. A pilot experiment with ChimeriVax™-DEN1-4 viruses as well as YF-VAX® was performed in advance to assure suitability of the cynomolgus monkeys as a replacement for rhesus.

Experiment 1

A total of 12 (6 males and 6 females), experimentally naive, flavivirus-seronegative rhesus monkeys, 2.7 to 4.3 years of age for males and 2.6 to 5.2 years of age for females, weighing 3.6 to 4.3 kg for males and 3.4 to 4.6 kg for females on the day prior to dosing, was assigned to 3 treatment groups (n=4). Each animal received a single dose (˜5 log₁₀ PFU/0.5 ml virus in Minimal Essential Medium (MEM) containing 50% fetal bovine serum (FBS)) of each of three viruses via SC injection: Group 1: ChimeriVax™-DEN1 (uncloned virus, Vero P4); Group 2: ChimeriVax™-DEN1 (clone E, Vero P6); and Group 3: ChimeriVax™-DEN1 PMS (clone J). The day of dosing was designated as Day 1. Blood samples were collected predose on Day 1 and on Days 2 through 11 for viremia analysis, and on Day 31 for neutralizing antibody analysis. Prior to assignment to the study, animals had been given a complete physical examination, including abdominal palpation and observations of the condition of integument, respiratory, and cardiovascular systems, as well as evaluation of a standard panel of serum chemistry and hematology parameters. Throughout the study, animals were observed for changes in general appearance and behavior (at least twice daily), body weight (weekly), and food consumption (daily). After the last sample collection on Day 31, all animals were returned to the SBi animal colony.

Experiment 2

ChimeriVax™-DEN1 PMS (clone J, P7) and ChimeriVax™-DEN1 VL (P10) (each 5 log₁₀ PFU), as well as YF-VAX® control vaccine (4.7 log₁₀ PFU), were administered (0.25 mL) by injection into the left frontal lobe of 18 experimentally naive, flavivirus-seronegative cynomolgus monkeys (n=6/group) prescreened to be seronegative to flaviviruses. Monkeys were kept under observation for 30 days post inoculation, then euthanized and necropsied. Selected tissues were removed from all animals and processed to slides prior to histopathological evaluation.

During the observation period, the monkeys were evaluated for changes in clinical signs (twice daily), body weight (weekly), and food consumption (daily). Clinical signs were assigned scores according to a clinical scoring system, based on the World Health Organization (WHO) requirements for yellow fever virus vaccines (World Health Organization, “Requirements for yellow fever vaccine,” Requirements for Biological Substances No. 3, revised 1995, WHO Tech. Rep. Ser. 872, Annex 2, Geneva: WHO, 31-68, 1998). Blood samples were collected pre-study and pre-inoculation on Day 1, and on Days 3, 5, 7, 15, and 31 for clinical pathology analysis (serum chemistry and hematology parameters). Additional blood samples were collected pre-inoculation on Day 1 and on Days 2-11 for viremia analysis, and on Days 1 (pre-dose) and 31 for measurement of neutralizing antibody response.

At necropsy, gross pathologic findings were recorded and a complete list of tissues was collected and preserved. Slides were prepared from a selected subset of tissues and examined for histopathologic findings by the Study Pathologist (liver, spleen, heart, kidney, and adrenal glands). Histopathology of the brain and spinal cord was performed by a neuropathologist according to WHO requirements for YF vaccines. The histopathological evaluation was performed in a blinded manner. Lesions in the meninges and the brain/spinal cord matter were scored using a scale of 0-2, according to the following observations: Grade 0: no visible lesions; Grade 1: (minimal), 1-3 small and/or one large infiltrate, mostly perivascular, a few small foci of more diffuse infiltration, unconnected with blood vessels; and Grade 2: (mild), more than 3 small and/or 2 or more, large perivascular infiltrates, several foci of cellular infiltration, unconnected with blood vessels (some neurons may be involved in these foci of inflammation). The degree of neurovirulence was estimated for the target and discriminator areas. For cynomolgus monkeys, the substantia nigra and cervical and lumbar enlargements of the spinal cord represent the target formations, whereas basal ganglia and thalamic nuclei are considered as discriminator areas. Individual and group mean lesion scores for the target and discriminator areas were calculated separately and as a combined score.

Plaque Assay

A standard plaque assay using Vero cells was performed on sera (undiluted or at 1:2 and 1:10 dilutions) obtained from Days 2-11 post infection. Viremia titers were expressed as PFU/ml. A plaque-reduction method using Vero cells was used for measurement of neutralizing antibody response to the homologous viruses (chimeras or YF-VAX). In this test, a constant virus input (˜50-100 PFU) is neutralized by varying serum dilutions (heat inactivated), and titers are expressed as the highest dilution of serum inhibiting 50% of the plaques (PRNT₅₀).

Growth Kinetics in HepG2 Cells

HepG2 cells were grown in Eagles MEM (Vitacell) supplemented with 8% FBS (Hyclone) and Antibiotic/Antimycotic (Sigma) to confluency in T25 flasks at 37° C. 5% CO₂, and infected at an MOI of 0.001 with ChimeriVax™-DEN1 PMS, ChimeriVax™-DEN1 VL, or the parent viruses (YF-VAX® and WT DEN1, strain PU0359) for 1 hour. Inocula were removed, cells were washed with PBS three times to remove unbound viruses, and growth medium was added to the cultures. Daily samples (10 days) were removed, FBS was added to a final concentration of 50% to preserve virus infectivity, and samples were stored at −70° C. Virus titers were determined by plaque assay on Vero cells using agarose double overlay and neutral red.

Mosquito Transmission

F4 generations of a laboratory established colony of Aedes aegypti from Puerto Rico were inoculated with ChimeriVax™-DEN1 PMS (P7), ChimeriVax™-DEN1 VL (P10), or control parent (YF 1 7D and WT DEN1, strain PUO359) viruses. Mosquitoes were cold anesthetized and inoculated intrathoracically (IT) to preclude the potential infection barriers in the midgut associated with oral feeding, using a microcapillary needle that had been pulled to a point with a Narishige (Tokyo) needle puller. Approximately 0.34 μl of virus standardized to 6.0 log₁₀ PFU/ml was injected into each mosquito (2.5 log₁₀ PFU/mosquito). Inoculated mosquitoes were maintained in cartons at 27° C., 80% humidity with 5% sugar water. Three mosquitoes per infection were removed at 48 hour intervals for 10 days; the remaining mosquitoes were collected at 14 days post-inoculation, and were frozen at −70° C. until assayed. Infectious virus titer was determined by real-time RT-PCR (TaqMan). Primers and probes were designed with the PrimerExpress software package (PE Applied Biosystems, Foster City, Calif.). The TaqMan probes were labeled at the 5′ end with the FAM reporter dye and at the 3′ end with the dark quencher dye. Each of the ChimeriVax™-DEN primers were serotype specific, whereas the YF 17D primers detected both ChimeriVax™-DEN and YF 17D viruses. Statistical Analyses

Differences in Average Survival Time (AST) among groups of suckling mice inoculated with DEN1 clones or YF-VAX® were analyzed for significance using Product-Limit Survival Fit. All other probability analyses for significance levels between two groups or among groups of animals were performed using Oneway Anova test. Observed significance probabilities of 0.050 or less are often considered evidence that an analysis of variance model fits the data. All analyses were performed using JMP software version 5.1.

Results

Neurovirulence Properties of Various Clones of DEN1 Chimeras in Suckling Mice

During plaque purification in the course of PMS production for DEN1 chimera, 10 different clones (A-J) were sequenced to identify a clone without any amino acid substitutions. All but 1 clone (J) contained 1 or 2 substitutions within the envelope protein E. Representative clones were evaluated for their neurovirulence using 4 day-old suckling mice inoculated by the IC route (Table 6). All clones, except clone E, exhibited similar neurovirulence with AST of 8.5 to 11.3 days, which was significantly higher than YF-VAX® (8.3 days). Clone E, which contained 2 mutations (one nucleotide change at1590 from A to G, resulting in a K to R substitution, and one nucleotide change at 3952 from A to T, which was silent), was significantly less virulent than all other DEN1 clones with an AST of 13-15 days. Interestingly, the only amino acid change identified on the E-protein of the original, uncloned DEN1 chimera was also the E204 K to R substitution. This virus had shown to induce a low level of viremia (mean peak titer 0.7 log₁₀ PFU/ml) for 1.3 days when inoculated into monkeys by the SC route (Guirakhoo et al., J. Virol. 75(16):7290-7304, 2001). Clone J, which did not contain any mutations and was shown to be significantly less virulent than YF-VAX® in 4 days old mice (see statistics in Table 6), was selected for production of the cGMP vaccine virus. To determine if attenuation of clone E for infant mice would correlate with a lower degree of viscerotropism/viremia and/or immunogenicity in monkeys, this clone and clone J (PMS) were inoculated into monkeys by SC route (see below).

Experiment 1. Viremia/Viscerotropism and Immunogenicity of ChimeriVax™DEN1 Viruses With or Without E204 Mutations in Monkeys Inoculated by the SC Route

Twelve rhesus flavivirus seronegative monkeys were divided into 3 groups (n=4). Animals in each group received a single dose (˜5 log₁₀ PFU virus/0.5 ml) of each 3 viruses via subcutaneous (SC) injection as shown in Table 7. During a 1 month observation period, no test article-related changes in clinical signs, food consumption or body weight were found.

Viremia and Neutralizing Antibody Response

As shown in Table 7, all 4 monkeys inoculated with DEN1 PMS virus (clone J, Group 3) became viremic, whereas 3/4 and 2/4 monkeys inoculated with clone E or uncloned DEN1 viruses, respectively, became viremic. Viremia was detected in all 4 animals of Group 3 until the last day of sample collection (Day 11), whereas no animal in Groups 1 and 2 was viremic beyond Day 5 (the level of detection 1 log₁₀ PFU/ml). The mean peak virus titers were 0.75 (1.5 for viremic animals), 1.3 (1.7 for viremic animals) and 2.5 log₁₀ PFU/ml for groups 1-3, respectively. The mean durations of viremia were 1 (2 for viremic animals), 1.5 (2 for viremic animals), and 8.5 days for groups 1-3, respectively. The magnitude and duration of viremia in Group 3 monkeys were significantly higher than those of Groups 1 and 2 (see statistics in Table 8) animals. Despite the lack of viremia in some monkeys, all animals developed neutralizing antibody titers against homologous viruses (Table 7). The geometric mean neutralizing antibody titers (GMT PRNT₅₀) were 538, 3620, and 8611 for Groups 1 to 3, respectively. Consistent with the level of viremia, the neutralizing titers in monkeys immunized with the PMS virus (Group 3, without mutation) were significantly higher than in the other 2 groups (Groups 1 and 2, with mutations) (see statistics in Table 7). The sera of Group 1 monkeys (immunized with a DEN1 chimera with 2 amino acid substitutions on the envelope proteins, M39 H>R and E204 K>R), revealed the lowest neutralizing titers.

Experiment 2. Safety/Neurovirulence of Chimeri Vax™-DEN1 Viruses With or Without the E204 Mutation in Monkeys Inoculated by the IC Route

Inoculation of suckling mice with clone E, containing a single amino acid substitution from K to R on the envelope protein E, had indicated that this site is involved in neurovirulence of DEN1 chimera for infant mice. Subsequently, when this clone was inoculated into monkeys by the SC route, it induced a significantly lower viremia in terms of magnitude and duration, compared to non-mutant virus (clone J PMS, P7). Interestingly, when clone J was passaged in Vero cells to produce the cGMP VL at P10, it acquired the same nucleotide change (nucleotide 1590 A to G, resulting in K to R substitution) as had been observed with clone E. The vaccine virus (P10) was similarly less virulent than the PMS (P7) when tested in infant mice. Since the attenuation of DEN1 vaccine (P10) was dependent on a single amino acid substitution on the E-protein (E204R), which theoretically could revert to the WT sequence (E204K) in a vaccinated individual, it was necessary to determine the safety profile of the non-mutant virus (WT envelope) when injected directly into the brain tissues.

Three groups of monkeys (n=6) were inoculated with ChimeriVax™-DEN1 PMS (P7, E204K), ChimeriVax™-DEN1 VL (P10, E204R), or YF-VAX® (as a control), by the IC routes. Animals were monitored for 31 days for clinical signs and then sacrificed for pathological evaluations.

Viremia

All 6 monkeys inoculated with ChimeriVax™-DEN1, PMS virus (Group 1) became viremic. The duration of viremia was generally 4-5 days with peak titers ranging from 1-3.3 log₁₀ PFU/mL (Table 9). The mean peak viremia was 2.5 log₁₀ PFU/mL, with the mean duration of 4.2 days (Table 10). Five of 6 monkeys inoculated with ChimeriVax™-DEN1 VL virus (Group 2) became viremic. The duration of viremia was generally 1-4 days with peak titers ranging from 1-2.1 log₁₀ PFU/mL (Table 9). The mean peak viremia was 1.4 (1.6 for viremic animals) log₁₀ PFU/mL, with a mean duration of 2.5 days (3 days for viremic animals) (Table 10).

All 6 monkeys inoculated with YF-VAX® (Group 3) became viremic. The duration of viremia was generally 2-4 days (with one exception, in which a viral titer of 1 log₁₀ PFU/ml was observed 9 days post inoculation following 4 days of undetectable titer) with peak titers ranging from 1-3 log₁₀ PFU/mL (Table 9). The mean peak viremia was 2.2 log₁₀ PFU/mL, and the mean number of viremic days was 2.8 days (Table 10). The peak titer and duration of viremia in Group 1 was significantly higher than Group 2. When Group 1 (P7) was compared with Group 3 (YF-VAX®), only the duration but not the magnitude of viremia was significant between the 2 groups. The viremia and duration of P 10 vaccine virus (Group 2) was similar to YF-VAX (for statistics see Table 10).

For all groups, monkey viremia titers were below 500 and 100 mouse IC LD₅₀ values (estimated to equal ˜20,000 and ˜4,000 Vero cell PFU/0.03 mL (Guirakhoo et al., Virology 257:363-372, 1999), respectively, for YF-VAX®), which are the maximum acceptable titers for individual monkey and group (i.e., present in no more than 10% of the monkeys) titers, respectively, as established under the WHO requirements for yellow fever 17D vaccine.

Immunogenicity

All monkeys seroconverted following treatment with YF-VAX®. On Day 31, PRNT₅₀ against YF virus ranged from 640 to 2560. One of the YF-VAX®-treated monkeys had cross-reactivity with heterologous DEN1 in a PRNT₅₀ assay on Day 31. Such antibody cross-reactivity is not unexpected among the flaviviruses. However, remote exposure of this monkey to a heterologous flavivirus that was not detected by pre-study antibody screening cannot be ruled out.

All monkeys seroconverted following treatment with ChimeriVax™-DEN1 PMS or ChimeriVax-DEN vaccine virus (Table 9). PRNT50 ranged from 1280-5120 and from 2560-10240 in the ChimeriVax™-DEN1 PMS and ChimeriVax™-DEN Vaccine treated groups, respectively, and no monkey had cross-reacting antibodies to YF 17D virus. Antibody levels varied inversely with viremia levels for both ChimeriVax™-DEN1 treated groups (see statistics in Table 9).

Histopathology

Minimal lesions were found in 1/6 monkeys inoculated with ChimeriVax™-DEN1 PMS virus and in 3/6 monkeys inoculated with ChimeriVax™-DEN1 VL (Table 11). Lesions were present in 5/6 monkeys inoculated with YF-VAX®. All of these lesions were inflammatory, with minimal and mild severity, and consisted of histiocytes and lymphocytes. Infiltration in the meninges was seen mainly around the site of inoculation. Scanty, mostly perivascular infiltrates were noted in the brain and/or spinal cord of positive monkeys. There was no involvement of the neurons in any animal. Lesions in the ChimeriVax™-DEN1-treated groups were generally minimal (grade 1), and grade 2 lesions were found in only one brain section of monkey F22205M that received ChimeriVax™-DEN1 VL. In the YF-VAX™-treated group, grade 2 lesions were present in a number of sections of the brains in four monkeys. The individual and group mean lesion scores for the target and discriminator areas and combined scores are shown in Table 11.

In non-central nervous system (CNS) tissues, the only vaccine-related histopathologic findings were minimal to mild splenic lymphoid hyperplasia in 4/6, 3/6, and 6/6 animals treated with ChimeriVax™-DEN1 (P7), ChimeriVax™-DEN1 (P10), or YF-VAX®, respectively. Lymphoid hyperplasia was considered secondary to immuno-stimulation in this study.

CNS lesions were observed in 1/6, 5/6, and 5/6 of monkeys inoculated with ChimeriVax™-DEN1 (P7), ChimeriVax™-DEN1 (P10), or YF-VAX®, respectively. All of these lesions were inflammatory with minimal and mild severity (grades 1 or 2). Scanty, mostly perivascular infiltrates were noted in the brain and/or spinal cord of those monkeys with lesions. There was no involvement of neurons in any animal. Lesions in the ChimeriVax™-DEN1-treated groups were generally minimal (grade 1), although one brain section of one monkey that received ChimeriVax™-DEN1 VL (P10) had a mild (grade 2) lesion. In the YF-VAX®-treated group, grade 2 lesions were present in a number of sections of the brain in 4 monkeys. Target-area, discriminator-area, and combined lesion scores for ChimeriVax™-DEN1 PMS virus and ChimeriVax™-DEN1 VL-treated groups were much lower than those for the reference YF-VAX®-treated group (see statistics in Table 11). The differences in target- and discriminator-areas lesion scores for the two ChimeriVax™DEN1 treated groups were not statistically significant (Table 11).

Growth Kinetics of ChimeriVax™DEN1 Virus With or Without the E204 Mutation in HepG2 Hepatoma Cells

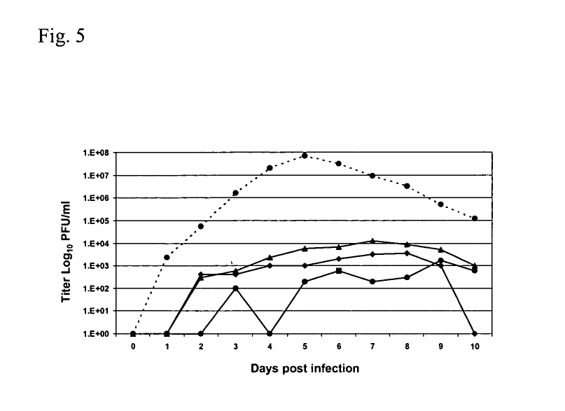

Both ChimeriVax™-DEN1 and parent WT DEN1 viruses grew slower and to significantly lower titers than the YF 17D virus. The peak titers were on Days 9, 8, 7, and 5 for WT DEN1, ChimeriVax™-DEN1 PMS (E204K), ChimeriVax™-DEN1 VL (E204R) and YF17D viruses, respectively. The virus concentrations at peak levels were ˜3.2, 3.6, 4.1, and 7.8 log₁₀ PFU/ml for WT DEN1, ChimeriVax™-DEN1 PMS, ChimeriVax™-DEN1 VL, and YF 17D viruses, respectively (FIG. 5).

Growth of Chimeri Vax™DEN1 Virus With or Without the E204 Mutation in Mosquitoes

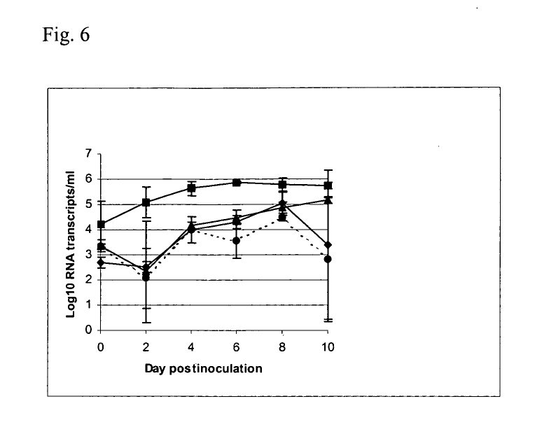

The rationale for this experiment was to assure that ChimeriVax™-DEN1 mutant vaccine will remain safe in the human host and will not replicate in mosquitoes even if it is reverted to WT sequence in a vaccinated individual. Replication and dissemination of ChimeriVax™-DEN1 viruses were evaluated in mosquitoes. Aedes aegypti mosquitoes were inoculated by the IT route with ChimeriVax™-DEN1 PMS (E204K P7), ChimeriVax™-DEN1 VL (E204R, P10), WT DEN1 (strain PU0359), or YF 17D viruses, and replication rates were compared. There were no significant differences between the two chimeric viruses and YF 17D. The WT DEN1 titer was about 0.5-2.5 logs higher than both of ChimeriVax™-DEN1 viruses (FIG. 6).

Position of Mutation 204 on the Crystal Structure of the DEN1 E Protein

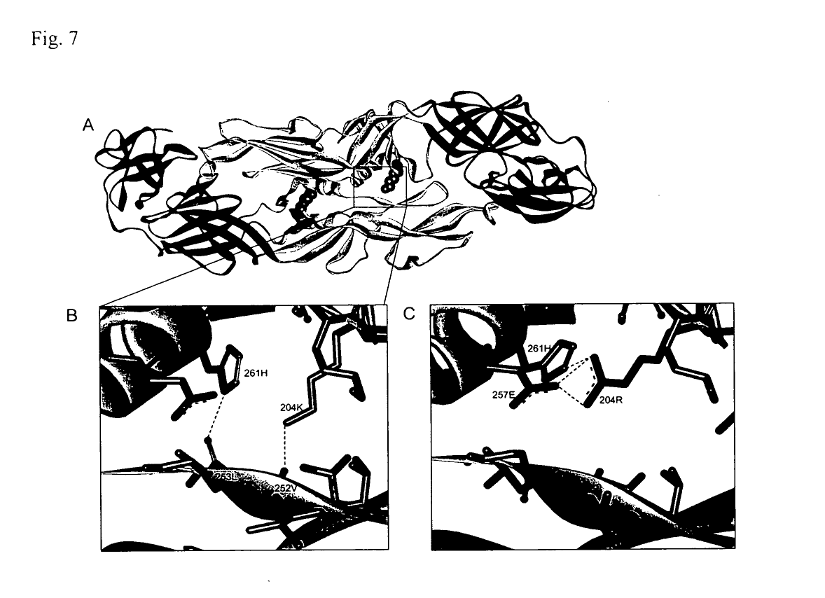

The structure of 394 residues of the DEN1 E protein ectodomain (strain PUO 359, representing the E-protein of ChimeriVax™-DEN1 (PMS, E204K, wt, p7)) was modeled based on the known structure of DEN2 virus (Modis et al., Proc. Natl. Acad. Sci. U.S.A. 100(12):6986-6991, 2003) using the homology modeling software from Accelrys (FIGS. 7A and 7B). The K residue at position 204 was mutated to R, and the modeling was repeated for the mutant virus to represent the E-protein structure of the ChimeriVax™-DEN1 (E204R, mutant, VL, P10) virus (FIG. 7C). Residue 204 is located within a short loop connecting the 2 beta strands f and g of the domain II (FIG. 7A) and is in proximity of the 2 alpha-helices, alpha-A and alpha-B. Domain II also carries the conserved fusion peptide in its tip. This short loop is located within a hydrophobic pocket lined by residues that influence neurovirulence or the pH threshold for viral fusion (Modis et al., Proc. Natl. Acad. Sci. U.S.A. 100(12):6986-6991, 2003). FIG. 7B is a close-up of the corresponding area in FIG. 7A with amino acid 204K shown in stick representation. The nitrogen (N) atoms of 204K and 261H side chains make H-bonds with oxygen (O) atoms of 252V (2.7 Å apart) and 253L (2.65 Å apart) side chains, respectively (FIG. 7B). In contrast, the mutation at 204 from K to R results in a conformation change in which the distances of 204R and 261H to 252V and 253L increase to 5.10 and 8.11 Å, respectively. This movement results in loss of intermolecular (between the 2 E-monomers) H-bonds between these residues. To compensate for the loss of these 2 H-bonds, the N atom of the 204R side chain in the mutant virus makes three new intramolecular (within the same E-monomer) bonds; between N of 204R and N and O atoms of 261H and 257E (3.01, 3.07, and 2.78 Å apart, respectively) (FIG. 7C). The interactions between R and E amino acids are probably salt bridges rather than H-bonds, since both of them are charged at neutral pH. Another interesting observation is that the side chain of 261H in mutant virus is flipped compared to its position in WT structure (compare 261H position in FIGS. 7B and 7C).

TABLE 1
Comparison of the amino acid differences in the E protein of ChimeriVax ™ -JE
FRhL₃ and Chimeri Vax ™ -JE FRhL₅ virus with published sequences
of JE SA14-14-2 vaccine, wild-type JE strains, parental SA14, and Nakayama
virus. ChimeriVax ™ -JE FRhL₃ and FRhL₅ viruses were sequenced across
their entire genomes and the mutation at E279 was the only difference found.

| | E | E | E | E | E | E | E | E | E | E |
|---|---|---|---|---|---|---|---|---|---|---|
| Virus | 107 | 138 | 176 | 177 | 227 | 244 | 264 | 279 | 315 | 439 |
| ChimeriVax ™ -JE FRhL₃ | F | K | V | A | S | G | H | M | V | R |
| E279 Met | | | | | | | | | | |
| ChimeriVax ™ -JE FRhL₅ | F | K | V | A | S | G | H | K | V | R |
| E279 Lys | | | | | | | | | | |
| JE SA14-14-2 PDK¹ | F | K | V | T | S | G | Q | M | V | R |
| JE SA14-14-2 PHK² | F | K | V | A | S | G | H | M | V | R |
| JE SA14^{1,3} | L | E | I | T | S | G | Q | K | A | K |
| JE Nakayama⁴ | L | E | I | T | P | E | Q | K | A | K |

¹Nitayaphan, et al., Virology 177: 541-552, 1990.
²Ni et al., J. Gen. Virol. 75: 1505-1510, 1994; PDK = primary dog kidney
³Aihara et al., Virus Genes 5: 95-109, 1991; PHK = primary hamster kidney
⁴McAda et al., Virology 158: 348-360, 1987.

TABLE 2
Neurovirulence for suckling mice of ChimeriVax ™ -JE
viruses with and without a mutation at E279 and YF 17D vaccine

| | Mouse | Virus, passage | Intracerebral | | Average | LD₅₀ |
|---|---|---|---|---|---|---|
| | age | and E279 amino | dose | Mortality | Survival | (Log₁₀ |
| Experiment | (days) | acid | (Log₁₀ PFU) | (%) | Time (Days) | PFU) |
| 1 | 8.6 | YF-VAX ® | 1.15 | 10/10 (100) | 8.4 | 0.11 |
| | | | 0.15 | 5/10 (50) | 10 | |
| | | | 0-0.85 | 1/10 (10) | 14 | |
| | | ChimeriVax ™ - | 2.60 | 1/10 (10) | 15 | >2.6 |
| | | JE, FRhL_{3,} | 1.6 | 1/10 (10) | 13 | |
| | | E279 Met | 0.6 | 0/10 (0) | N/A | |
| | | | −0.45 | 0/10 (0) | N/A | |
| | | ChimeriVax ™ - | 3.0 | 10/10 (100) | 10.3 | 1.64 |
| | | JE, FRhL_{5,} | 2.0 | 8/10 (80) | 11.25 | |
| | | E279 Lys | 1.0 | 2/10 (20) | 14.5 | |
| | | | 0 | 2/10 (20) | 16 | |
| 2 | 4 | YF-VAX ® | 0.95 | 11/11 (100) | 8.4 | −0.3 |
| | | | −0.05 | 9/11 (82) | 8.8 | |
| | | | −1.05 | 2/12 (17) | 10 | |
| | | ChimeriVax ™ - | 2.69 | 7/12 (58) | 10.6 | 2.5 |
| | | JE, FRhL_{3,} | 1.69 | 4/12 (33) | 11.5 | |
| | | E279 Met | 0.69 | 0/12 (0) | NA | |
| | | ChimeriVax ™ - | 2.88 | 10/12 (83) | 9.3 | 1.45 |
| | | JE, FRhL_{5,} | 1.88 | 11/12 (92) | 10.3 | |
| | | E279 Lys | 0.88 | 4/12 (33) | 12.2 | |
| | | | −0.11 | 2/12 (17) | 14 | |
| | | | −1.11 | 0/12 (0) | NA | |
| | | YF/JE₂₇₉ site- | 3.55 | 12/12 (100) | 9.4 | 1.15 |
| | | specific | 2.55 | 11/12 (92) | 10.1 | |
| | | revertant, | 1.55 | 11/12 (92) | 10.2 | |
| | | E279 Lys | 0.55 | 3/12 (25) | 10.7 | |
| | | | −0.44 | 2/12 (17) | 14 | |

TABLE 3
Neuropathological evaluation of monkeys inoculated IC with Chimeri
Vax ™ -JE FRhL_{3,} FRhL_{5,} or yellow fever 17D
(YF-VAX ®) and necropsied on day 30 post-inoculation.

| | | | | Clinical | | | |
|---|---|---|---|---|---|---|---|
| | | | Dose¹ | score² | Individual and group mean | | |
| | | | log₁₀ | Maximum | histopathological score | | |
| | | | PFU/ | score/Mean | Target | Discriminator | Target + |
| Test virus | Monkey | Sex | 0.25 mL | daily score | area³ | areas⁴ | Discriminator areas |
| YF-VAX ® | RT702M | M | 4.05 | 1/0 | 2.00 | 0.51 | 1.26 |
| Connaught | RT758M | M | 4.28 | 1/0 | 0.25 | 0.01 | 0.13 |
| Lot # 0986400 | RT653M | M | 4.07 | 1/0 | 2.00 | 0.39 | 1.20 |
| | RT776M | M | 4.25 | 3/1 | 2.00 | 1.29 | 1.65 |
| | RT621M | M | 4.34 | 3/2 | 1.00 | 0.46 | 0.73 |
| | RAH80F | F | 4.14 | 3/1 | 1.50 | 0.71 | 1.10 |
| | RAL02F | F | 4.13 | 1/1 | 2.00 | 0.80 | 1.40 |
| | RT698F | F | 3.78 | 3/1 | 1.50 | 0.64 | 1.07 |
| | RAI12F | F | 4.11 | 1/1 | 2.00 | 1.45 | 1.73 |
| | RP942F | F | 4.05 | 1/0 | 2.00 | 0.81 | 1.41 |
| | Mean | | 4.12 | 1 | 1.63 | 0.71 | 1.17 |
| | SD | | 0.16 | 1 | 0.59 | 0.42 | 0.47 |
| ChimeriVax ™ - | RT452M | M | 3.55 | 1/0 | 0.50 | 0.08 | 0.29 |
| JE, FRhL₃ | RR257M | M | 3.52 | 1/0 | 1.00 | 0.14 | 0.57 |
| Lot # I031299A | RT834M | M | 3.71 | 1/0 | 0.50 | 0.38 | 0.44 |
| | RT620M | M | 3.71 | 1/0 | 1.00 | 0.14 | 0.57 |
| | RT288M | M | 3.76 | 1/0 | 0.50 | 0.19 | 0.35 |
| | RAJ98F | F | 3.79 | 1/1 | 0.00 | 0.11 | 0.05 |
| | RAR08F | F | 3.52 | 1/0 | 0.00 | 0.13 | 0.07 |
| | RV481F | F | 3.52 | 1/0 | 0.00 | 0.06 | 0.03 |
| | RT841F | F | 3.71 | 1/0 | 0.50 | 0.05 | 0.28 |
| | RT392F | F | 3.76 | 1/0 | 0.50 | 0.07 | 0.29 |
| | Mean | | 3.66 | 0 | 0.45 | 0.14 | 0.29 |
| | SD | | 0.11 | 0 | 0.37 | 0.10 | 0.20 |
| P-value (t Test⁵) vs. YF-VAX ® | | | | 0.037/0.025 | 0.00008 | 0.00191 | 0.00014 |
| ChimeriVax ™ - | RT628M | M | 4.20 | 1/0 | 0.50 | 0.57 | 0.54 |
| JE, FRhL₅ | RT678M | M | 4.19 | 1/0 | 1.00 | 0.12 | 0.60 |
| Lot # 99B01 | RT581M | M | 4.17 | 1/0 | 1.00 | 0.46 | 0.73 |
| | RR726M | M | 4.32 | 1/0 | 1.00 | 0.66 | 0.83 |
| | RR725M | M | ND⁶ | 1/0 | 1.00 | 0.33 | 0.67 |
| | RAJ55F | F | 4.27 | 0/0 | 1.00 | 0.14 | 0.57 |
| | RT769F | F | 4.44 | 1/0 | 1.00 | 0.58 | 0.79 |
| | RAK22F | F | 4.24 | 1/0 | 0.00 | 0.12 | 0.06 |
| | RT207F | F | 4.49 | 1/1 | 1.00 | 0.22 | 0.61 |
| | RT490F | F | 4.34 | 1/0 | 0.00 | 0.04 | 0.02 |
| | Mean | | 4.30 | 0 | 0.75 | 0.32 | 0.54 |
| | SD | | 0.11 | 0 | 0.42 | 0.23 | 0.28 |

| | | | | |
|---|---|---|---|---|
| P-value (t Test) vs. YF-VAX ® | 0.024/0.025 | 0.00154 | 0.02436 | 0.00248 |
| P-value (t Test) vs. ChimeriVax ™ -JE FRhL₃ | 0.343/1.00 | 0.10942 | 0.03223 | 0.03656 |

¹Back-titration
²Clinical score: 0 = no signs; 1 = rough coat, not eating; 2 = high pitched voice, inactive, slow moving; 3 = tremor, incoordination, shaky movements, limb weakness; 4 = inability to stand, paralysis, moribund, or dead. The maximum score on any day and the mean score over the 30-day observation period are shown.
³Substantia nigra
⁴Corpus striatum and thalamus, right and left side (N. caudatus, globus pallidus, putamen, N. ant./lat. thalami, N. at. thalami; cervical and lumbar enlargements of the spinal cord (6 levels)
⁵Student's t test, two-sided, heteroscedastic, comparing YF-VAX ® and ChimeriVax ™ -JE viruses.
⁶Not done

TABLE 4
Viremia, rhesus monkeys inoculated IC with YF-VAX ® or
ChimeriVax ™ -JE FRHL₃ and FRHL₅ viruses
(for dose inoculated, see Table 3).
YF-VAX ® Control

| | Serum Virus Titer (Log₁₀ PFU/mL), Day | | | | | | | | |
|---|---|---|---|---|---|---|---|---|---|
| Animal | 1 | 2 | 3 | 4 | 5 | 6 | 7 | 8 | 9 |
| RT702M | —¹ | — | 1.6 | 3.0 | — | — | — | — | — |
| RAH80F | — | — | — | 3.3 | 2.5 | — | — | — | — |
| RT758M | — | — | 2.1 | 3.2 | 2.8 | — | — | — | — |
| RAL02F | — | — | — | 1.3 | — | — | — | — | — |
| RT653M | — | — | — | 2.7 | — | — | — | — | — |
| RT698F | — | 1.0 | 2.3 | 3.7 | 2.5 | — | 1.0 | — | — |
| RT776M | — | — | — | — | — | — | — | — | — |
| RAI12F | — | — | — | 2.0 | 2.5 | 2.5 | 2.0 | — | — |
| RT621M | — | 1.0 | 2.0 | 3.3 | 2.0 | — | — | — | — |
| RP942F | — | 1.0 | 2.6 | 3.6 | 2.0 | — | — | — | — |
| Mean Titer² | | 0.8 | 1.4 | 2.7 | 1.7 | 0.9 | 0.9 | | |
| SD | | 0.1 | 0.8 | 1.0 | 0.9 | 0.6 | 0.4 | | |

ChimeriVax ™ -JE FRHL₃ E279 Met

| | Serum Virus Titer¹ (Log₁₀ PFU/mL), Day | | | | | | | | |
|---|---|---|---|---|---|---|---|---|---|
| Animal | 1 | 2 | 3 | 4 | 5 | 6 | 7 | 8 | 9 |
| RAJ98F | — | — | 1.9 | 1.3 | — | — | — | — | — |
| RT452M | — | 1.3 | 2.1 | 1.6 | — | — | — | — | — |
| RAR08F | — | — | 1.3 | 2.2 | 2.2 | 1.8 | — | — | — |
| RR257M | — | — | 1.9 | 2.2 | 1.8 | — | — | — | — |
| RV481F | — | — | 2.1 | 1.8 | 1.5 | — | — | — | — |
| RT834M | — | — | 2.5 | 1.3 | — | — | — | — | — |
| RT841F | — | — | 2.4 | 1.7 | — | — | — | — | — |
| RT620M | — | — | 1.6 | 1.0 | — | — | — | — | — |
| RT392F | — | — | — | — | — | — | — | — | — |
| RT288M | — | — | — | — | — | — | — | — | — |
| Mean Titer | | 0.8 | 1.7 | 1.5 | 1.0 | 0.8 | | | |
| SD | | 0.2 | 0.6 | 0.5 | 0.6 | 0.3 | | | |
| P-value³ | | 0.696 | 0.386 | 0.003 | 0.065 | 0.745 | | | |

ChimeriVax ™ -JE FRhL₅ E279 Lys

| | Serum Virus Titer¹ (Log PFU/mL), Day | | | | | | | | |
|---|---|---|---|---|---|---|---|---|---|
| Animal | 1 | 2 | 3 | 4 | 5 | 6 | 7 | 8 | 9 |
| RT628M | — | — | — | — | — | — | — | — | — |
| RAJ55F | — | — | — | — | — | — | — | — | — |
| RT678M | — | — | — | — | — | — | — | — | — |
| RT769F | — | — | — | 2.0 | — | — | — | — | — |
| RT581M | — | — | — | — | — | — | — | — | — |
| RAK22F | — | — | — | — | — | — | 1.8 | — | — |
| RR726M | — | — | — | — | — | — | — | — | — |
| RT207F | — | — | — | — | — | — | — | — | — |
| RR725M | — | — | — | — | — | — | — | — | — |
| RT490F | — | — | — | — | — | — | — | — | — |
| Mean Titer | | 0.7 | 0.7 | 0.8 | 0.7 | 0.7 | 0.8 | | |
| SD | | 0.0 | 0.0 | 0.4 | 0.0 | 0.0 | 0.4 | | |
| P-value⁴ | | 0.331 | <0.000 | 0.010 | 0.076 | 1.0 | 1.0 | | |

—¹ = No detectable viremia; in most tests neat serum was tested, the cutoff being 1.0 log₁₀ PFU/mL); in some cases, neat serum was toxic to cells, and serum diluted 1:2 or 1:5 was used (cut-off 1.3 or 1.7 log₁₀ PFU/mL).
²For the purpose of calculating mean titers and standard deviations, 0.7 was used in place of <1.0, 1.0 was used in place of <1.3, and 1.4 was used in place of <1.7.
³Comparison with YF-VAX ® by t-test, 2-tailed
⁴Comparison with ChimeriVax ™ JE FRhL₃ by t-test, 2-tailed

TABLE 5
Nucleotide and amino acid sequences of uncloned and various clones of ChimeriVax-DEN1
viruses and their in vitro (Vero passages) genetic stabilities.

| | | | | Nt. change/ | AA change/ | | |
|---|---|---|---|---|---|---|---|
| Virus | Passage | Gene | Nt. No^{a} | heterogeneity | heterogeneity | AA No^{b} | comments |
| Uncloned | P2 | — | — | — | — | — | No mutations |
| Uncloned | P5 | E | 1590 | A/G | K/R | 204 | Nucleotide heterogeneity |
| | | E | 1730 | G/T | V/F | 251 | Nucleotide heterogeneity |
| | | E | 1912 | G/t | E/D | 311 | Barely detectable mutant |
| | | E | 2282 | C/a | L/I | 435 | Undetectable mutants in some |
| | | | | | | | samples |
| Uncloned | P15 | E | 1590 | A to G | K to R | 204 | |
| | | NS2B | 4248 | G to T | G to V | | |
| | | NS4A | 6888 | C/T | A/V | | Nucleotide heterogeneity |
| | | NS4A | 7237 | A/G | I/M | | Nucleotide heterogeneity |
| Uncloned | P15 | E | 1590 | A to G | K to R | 204 | |
| | REPEAT | E | 1730 | G/T | V/F | 251 | Nucleotide heterogeneity |
| | from P2 | NS4A | 7237 | A/G | I/M | 263 | Nucleotide heterogeneity |
| | | NS4B | 7466 | C/t | P/S | 52 | Barely detectable mutant |
| Clone A | P3, P7 | E | 1730 | G to T | V to F | 251 | Domain II j strand, no function assigned |
| | | E | 2282 | C to A | L to I | 435 | Before anchor; L and I in D2 and YF |
| | | | | | | | respectively. (a gap left, nt 7080-7220) |
| Clone B | P3, P7, P10 | E | 1730 | G to T | V to F | 251 | |
| Clone C | P3, P6 | E | 1912 | G to T | E to D | 311 | Domain III, a strand, no function |
| | | | | | | | assigned. |
| Clone D | P3, P6 | E | 1730 | G to T | V to F | 251 | |
| Clone E | P3, P6 | E | 1590 | A to G | K to R | 204 | Domain II, f-g loop of, no function |
| | | | | | | | ass. |
| Clone F | P3 | M | 788 | C to T | — | — | |
| | | E | 1590 | A to G | K to R | 204 | |
| Clone G | P3 | E | 1730 | G to T | V to F | 251 | |
| Clone H | P3 | E | 1912 | G to T | E to D | 311 | |
| | | E | 2030 | G to T | V to L | 351 | Domain III, d strand (L in D2 and D3; I |
| | | | | | | | in D4) |
| Clone I | P3 | E | 1590 | A to G | K to R | 204 | |
| Clone J | P3, P6, P7, | — | — | — | — | — | |
| (J-2) | P10 | | | | | | |
| Cline J | P8 | E | 1590 | A to G (a/G) | K to R | 204 | Some parent (a) nucleotide still |
| (J-2) | (cGMP MS) | | | | | | present |
| | P10 from | E | 1590 | A to G | K to R | 204 | |
| | (cGMP MS) | | | | | | |
| Clone J | P10 REPEAT | E | 1590 | A to G | K to R | 204 | |
| (J-2) | from P7 | | | | | | |
| Clone J | P20 From | E | 1590 | A to G | K to R | 204 | |
| (J-2) | P10 repeat | NS4A | 6966 | G/T | S/I | 171 | |
| | | NS4A | 7190 | G/a | V/I | 246 | |

^{a}From the beginning of the genome.
^{b}From the N-terminus of indicated protein; numbering according to Rice et al., Science 229: 726-733, 1985. Clones with 204 mutations are shown in bold letters.

TABLE 6
Neurovirulence of various clones of ChimeriVax ™ -DEN1
viruses in 4-day old mice inoculated by the IC route

| | ChimeriVax ™ - | AA | | Dose | No dead/total | AST^{c} |
|---|---|---|---|---|---|---|
| Group | DEN1 | Change | Dilution | (BT)^{b} | (% dead) | Days |
| 1 | Uncloned | None | Neat | 5.0 | 11/11 (100) | 9.1 |
| | | | 1:10 | 4.1 | 11/11 (100) | 10.2 |
| 2 | Clone B | E251 (V > F) | Neat | 5.8 | 10/11 (91) | 9.8 |
| | | | 1:10 | 5.0 | 11/11 (100) | 10.2 |
| 3 | Clone C | E311 (E > D) | Neat | 5.8 | 11/11 (100) | 8.5 |
| | | E351 (V > L) | 1:10 | 4.9 | 11/11 (100) | 9.5 |
| 4 | Clone E | E204 (K > R) | Neat | 5.9 | 3/11 (27) | 13 |
| | | | 1:10 | 4.8 | 1/11 (9) | 14 |
| | | | 1:100 | 4.0 | 1/11 (9) | 15 |
| 5 | Clone J | None | Neat | 3.6 | 11/11 (100) | 10.8 |
| | | | 1:10 | 3.0 | 11/11 (100) | 11.3 |
| | | | 1:100 | 1.8 | 9/11 (82) | 11.3 |
| 6 | YE-VAX ® | NA | 1:20 | 2.5 | 12/12 (100) | 8.3 |

| | |
|---|---|
| Statistics (Probability)^{a} | |
| Group 1, 2, 3, 5 vs. Group 6 | 0.001 |
| Group 1, 2, 3, 5 vs. Group 4 | 0.0001 |
| Group 5 vs. Group 6 | 0.001 |

^{a}P values shown in bold numbers are considered statistically significant.
^{b}back titration.
^{c}Average Survival Time.

TABLE 7
Viremia and neutralizing antibody responses in monkeys inoculated SC with
5 log₁₀ PFU/0.5 ml of each ChimeriVax ™ -DEN1 viruses

| | | | Viremia (log₁₀ PFU/ml) | | | | | | | | | | |
|---|---|---|---|---|---|---|---|---|---|---|---|---|---|
| | | Virus | by post-immunization day^{b}: | | | | | | | | | | PRNT₅₀ |
| Group | Monkey | (AA change) | 2 | 3 | 4 | 5 | 6 | 7 | 8 | 9 | 10 | 11 | Day 31 |
| 1 | R18265M | Uncloned | —^{c} | — | — | — | — | — | — | — | — | — | 640 |
| | R175110F | (M39 H > R, | — | — | — | 1.7 | — | — | — | — | — | — | 640 |
| | F17572M | E204 K > R) | 1.3 | 1.0 | — | 1.0 | — | — | — | — | — | — | 320 |
| | F171114F | | — | — | — | — | — | — | — | — | — | — | 640 |
| GMT | | | | | | | | | | | | | 538 |
| 2 | R182103M | Clone E | — | — | — | — | — | — | — | — | — | — | 5120 |
| | R17098F | (E204 K > R) | — | 1.7 | — | — | — | — | — | — | — | — | 2560 |
| | R18261M | | 1.7 | 2.5 | 1.3 | 2.0 | | | | | | — | 2560 |
| | R175118F | | — | — | 1.0 | — | — | — | — | — | — | — | 5120 |
| GMT | | | | | | | | | | | | | 3620 |
| 3 | R182104M | Clone J, | 1.0 | 1.9 | 1.7 | 1.7 | 1.8 | 1.7 | 1.0 | 1.0 | 1.7 | — | 5120 |
| | R175108F | PMS, P7 | — | 1.7 | 2.8 | 2.2 | 1.0 | 2.0 | 1.7 | 2.0 | 2.2 | 1.7 | 10240 |
| | R182111M | (none) | 2.3 | 3.0 | 3.3 | 2.8 | 1.7 | 1.7 | — | — | — | — | 10240 |
| | R175104F | | — | 2.4 | 1.3 | 2.0 | 2.3 | 1.7 | 1.7 | 2.2 | 3.0 | 3.1 | 10240 |
| GMT | | | | | | | | | | | | | 8611 |

| | |
|---|---|
| Statistics (Probability)^{a} | |
| Group 1 vs. Group 2 | 0.0045 |
| Group 1 vs. Group 3 | 0.0006 |
| Group 2 vs. Group 3 | 0.0130 |

^{a}P values shown in bold numbers are considered statistically significant.
^{b}Monkeys were immunized on Day 1.
^{c}<1.0 log₁₀ PFU/ml.

TABLE 8
Summary of viremia shown in Table 7

| | | | Mean | |
|---|---|---|---|---|
| | Virus | No. Viremic/ | Peak titer | Duration |
| Group | (AA change) | no. tested (%) | Log₁₀ PFU/ml | (Days) |
| 1 | Uncloned | 2/4 (50) | 0.75 (1.5)^{b} | 1 (2)^{b} |
| | (M39 H > R and | | | |
| | E204 K > R) | | | |
| 2 | Clone E | 3/4 (75) | 1.3 (1.7)^{b} | 1.5 (2)^{b} |
| | (E204 K > R) | | | |
| 3 | Clone J, PMS, | 4/4 (100) | 2.5 | 8.5 |
| | P7 (none) | | | |

| | | |
|---|---|---|
| Statistics (Probability)^{a} | | |
| Group 1 vs. Group 2 | 0.45 | 0.67 |
| Group 1 vs. Group 3 | 0.009 | 0.0004 |
| Group 2 vs. Group 3 | 0.053 | 0.001 |

^{a}P values shown in bold numbers are considered statistically significant.
^{b}Viremic animals only.

TABLE 9
Viremia and neutralizing antibody responses in monkeys following IC inoculation
with ChimeriVax ™ -DEN1 PMS or ChimeriVax ™ -DEN 1 VL
viruses (5 log₁₀ PFU/0.25 ml, each) or with YE-VAX ® (4.7 log₁₀ PFU/0.25 ml).

| | | | Viremia (log₁₀ PFU/ml) by | | | | | | | | | | |
|---|---|---|---|---|---|---|---|---|---|---|---|---|---|
| | | Virus | post-immunization day^{b}: | | | | | | | | | | PRNT₅₀ |
| Group | Monkey | (AA change) | 2 | 3 | 4 | 5 | 6 | 7 | 8 | 9 | 0 | 11 | 31 |
| 1 | F22220M | ChimeriVax ™ - | —^{c} | 1 | 1 | 1.3 | 1.3 | 1 | — | — | — | — | 1280 |
| | F22236M | DEN1, | 3.3 | — | 2.2 | 2.3 | 1 | — | — | — | — | — | 5120 |
| | F22240M | PMS, P7 | 2.1 | 2.5 | 1.9 | 1.7 | — | — | — | — | — | — | 1280 |
| | F22276F | (none) | 2.2 | 1.8 | 1 | 1 | — | — | — | — | — | — | 1280 |
| | F22282F | | 1.7 | 3.1 | 2.9 | 2.5 | — | — | — | — | — | — | 1280 |
| | F222106F | | 1.6 | — | 2.7 | 2.8 | 2.6 | — | — | — | — | — | 1280 |
| GMT₅₀ | | | | | | | | | | | | | 1613 |
| 2 | F22203M | ChimeriVax ™ - | 1 | — | 1.7 | 1.5 | 1.3 | — | — | — | — | — | 10240 |
| | F22205M | DEN1, | 2.1 | 1.8 | 1.3 | 1.3 | — | — | — | — | — | — | 2560 |
| | F22246M | VL, P10 | 1.8 | 1.8 | 1.3 | 1 | — | — | — | | | — | 10240 |
| | F22287F | (E204 K > R) | — | — | 1.6 | — | 1 | — | — | — | — | — | 2560 |
| | F222112F | | 1 | — | — | — | — | — | — | — | — | — | 10240 |
| | F222115F | | — | — | — | — | — | — | — | — | — | — | 2560 |
| GMT₅₀ | | | | | | | | | | | | | 5120 |
| 3 | F22200M | YE-VAX ® | 1.9 | 2.3 | 2.6 | | | — | — | — | — | — | 1280 |
| | F22239M | | 1 | 1.3 | 1.3 | 1.7 | — | — | — | — | 1 | — | 640 |
| | F22256M | | — | — | 1.8 | 2.2 | — | — | — | — | — | — | 2560 |
| | F22280F | | — | 1 | | 1 | — | — | — | — | — | — | 1280 |
| | F22291F | | 1 | 1.7 | 2.8 | — | — | — | — | — | — | — | 1280 |
| | F22292F | | 1.3 | 3 | — | — | — | — | — | — | — | — | 2560 |
| GMT₅₀ | | | | | | | | | | | | | 1600 |

| | |
|---|---|
| Statistics (Probability)^{a} | |
| Group 1 vs. Group 2 | 0.016 |
| Group 1 vs. Group 3 | 0.664 |
| Group 2 vs. Group 3 | 0.021 |

^{a}P values shown in bold numbers are considered statistically significant.
^{b}Monkeys were inoculated on Day 1.
^{c}<1.0 log₁₀ PFU/ml.

TABLE 10
Summary of viremia shown in Table 9

| | | | Mean | |
|---|---|---|---|---|
| | Virus | No. Viremic/ | Peak titer | Duration |
| Group | (AA change) | no. tested (%) | Log₁₀ PFU/ml | (Days) |
| 1 | ChimeriVax ™ - | 6/6 (100) | 2.5 | 4.2 |
| | DEN1, PMS, | | | |
| | P7 (None) | | | |
| 2 | ChimeriVax ™ - | 5/6 (83) | 1.4 (1.6)b | 2.5 (3)^{b} |
| | DEN1, VL, P10 | | | |
| | (E204K > R) | | | |
| 3 | YF-VAX ® | 6/6 (100) | 2.2 | 2.8 |

| | | |
|---|---|---|
| Statistics (Probability)^{a} | | |
| Group 1 vs. Group 2 | 0.021 | 0.047 |
| Group 1 vs. Group 3 | 0.47 | 0.025 |
| Group 2 vs. Group 3 | 0.081 | 0.71 |

^{a}P values shown in bold numbers are considered statistically significant.
^{b}Viremic animals only

TABLE 11
Histopathological evaluation (lesion scores) of brains and spinal
cords in monkeys following IC inoculation with ChimeriVax ™ -
DEN1 PMS or ChimeriVax ™ -DEN 1 VL viruses (5 log₁₀
PFU/0.25 ml, each) or YF-VAX ® (4.7 log₁₀ PFU/0.25 ml)

| Group | Monkey | Target | Discriminator | Combined |
|---|---|---|---|---|
| (Virus) | Number | Areas | Area | Scores |
| 1 | F22220M | 0 | 0 | 0 |
| (ChimeriVax ™ - | F22236M | 0 | 0 | 0 |
| DEN1, PMS) | F22240M | 0 | 0 | 0 |
| | F22276F | 0 | 0 | 0 |
| | F22282F | 0.03 | 0.06 | 0.045 |
| | F222106F | 0 | 0 | 0 |
| Mean (SD) | | 0.01 (0.01) | 0.01 (0.02) | 0.01 (0.02) |
| 2 | F22203M | 0 | 0.06 | 0.03 |
| (ChimeriVax ™ - | F22205M | 0.08 | 0.31 | 0.195 |
| DEN1, VL) | F22246M | 0 | 0.06 | 0.03 |
| | F22287F | 0.17 | 0 | 0.085 |
| | F222112F | 0 | 0 | 0 |
| | F222115F | 0.20 | 0 | 0.10 |
| Mean (SD) | | 0.075 (0.091) | 0.072 (0.120) | 0.073 (0.070) |
| 3 | F22200M | 0 | 0 | 0 |
| (YE-VAX ®) | F22239M | 0.17 | 0.69 | 0.43 |
| | F22256M | 0.72 | 1.54 | 1.13 |
| | F22280F | 0.22 | 0.50 | 0.36 |
| | F22291F | 0.53 | 0.25 | 0.39 |
| | F22292F | 0.61 | 1.13 | 0.87 |
| Mean (SD) | | 0.38 (0.28) | 0.69 (0.57) | 0.53 (0.4) |
| Statistics (Probability)^{a} | | | | |
| Group 1 vs. Group 2 | | 0.092 | 0.25 | 0.0055 |
| Group 1 vs. Group 3 | | 0.009 | 0.016 | 0.010 |
| Group 2 vs. Group 3 | | 0.034 | 0.027 | 0.021 |

^{a}P values shown in bold numbers are considered statistically significant.

1. A flavivirus comprising a hinge region mutation that attenuates the flavivirus.

2. The flavivirus of claim 1, wherein the mutation decreases the viscerotropism of the flavivirus.

3. The flavivirus of claim 1, wherein the flavivirus comprises a yellow fever virus vaccine strain.

4. The flavivirus of claim 1, wherein the flavivirus is a viscerotropic flavivirus selected from the group consisting of Dengue virus, West Nile virus, Wesselsbron virus, Kyasanur Forest Disease virus, and Omsk Hemorrhagic fever virus.

5. The flavivirus of claim 1, wherein the flavivirus is a chimeric flavivirus.

6. The flavivirus of claim 5, wherein the chimeric flavivirus comprises the capsid and non-structural proteins of a first flavivirus virus and the pre-membrane and envelope proteins of a second flavivirus comprising an envelope protein mutation that attenuates the chimeric flavivirus.

7. The flavivirus of claim 6, wherein the second flavivirus is a Japanese encephalitis virus.

8. The flavivirus of claim 6, wherein the second flavivirus is a Dengue virus.

9. The flavivirus of claim 8, wherein the Dengue virus is Dengue-1, Dengue-2, Dengue-3, or Dengue-4 virus.

10. The flavivirus of claim 1, wherein the mutation is in the hydrophobic pocket of the hinge region of the envelope protein.

11. The flavivirus of claim 10, wherein the second flavivirus is a Dengue virus and the mutation is in the lysine at Dengue envelope amino acid position 202 or 204.

12. The flavivirus of claim 11, wherein the mutation is a substitution of the lysine.

13. The flavivirus of claim 12, wherein the lysine is substituted with arginine.

14. A vaccine composition comprising the flavivirus of claim 1 and a pharmaceutically acceptable carrier or diluent.

15. A method of inducing an immune response to a flavivirus in a patient, the method comprising administering to the patient the vaccine composition of claim 14.

16. The method of claim 15, wherein the patient does not have, but is at risk of developing, infection by the flavivirus.

17. The method of claim 15, wherein the patient is infected by the flavivirus.

18. A method of producing a vaccine comprising a flavivirus, the method comprising introducing into the flavivirus a mutation that results in decreased viscerotropism.

19. The method of claim 18, wherein the mutation is in the hinge region of the envelope protein of the flavivirus.

20. The method of claim 19, wherein the mutation is in the hydrophobic pocket of the envelope protein of the flavivirus.

21. A method of identifying a flavivirus vaccine candidate, the method comprising the steps of:
introducing a mutation into the hinge region of the flavivirus; and
determining whether the flavivirus comprising the hinge region mutation has decreased viscerotropism, as compared with a flavivirus virus lacking the mutation.

22. The method of claim 21, wherein the mutation is in the hinge region of the envelope protein of the flavivirus.

23. The method of claim 21, wherein the flavivirus is a yellow fever virus.

24. The method of claim 21, wherein the flavivirus is a chimeric flavivirus.
